(19) United States
(12) Patent Application Publication
(10) Pub. No.: US 2001/0049111 A1
(43) Pub. Date: Dec. 6, 2001

(54) Methods, procedures, and formats for using microelectronic array devices to perform multiplex immunoassay analyses

(76) Inventors: Norbert Windhab, Hofheim (DE); Michael J. Heller, Encinitas, CA (US); Richard R. Anderson, Encinitas, CA (US); Michael D. Fiechtner, Poway, CA (US); Tina S. Nova, Rancho Santa Fe, CA (US); Markus Schweitzer, Frankfurt am Main (DE); Alfred R. Sundquist, San Diego, CA (US); Christoph Brucher, Sulzbach (DE); Jill M. Orwick, San Diego, CA (US); Jochen Muller, Diez (DE); Stefan Raddatz, Wiesbaden (DE); Donald E. Ackley, Cardiff, CA (US); Christian Hamon, Frankfurt am Main (DE)

Correspondence Address: LYON & LYON LLP, 633 WEST FIFTH STREET, SUITE 4700, LOS ANGELES, CA, 90071, US

(21) Appl. No.: 09/783,763

(22) Filed: Feb. 14, 2001

Related U.S. Application Data
(63) Continuation-in-part of application No. 09/374,338, filed on Aug. 13, 1999.

(51) Int. Cl. G01N 33/53

(52) U.S. Cl. 435/7.1; 435/7.5

(57) ABSTRACT

This invention relates to devices and methods for carrying out multi-step and multiplex immunoaffinity binding reactions in microscopic formats. In particular, these devices and methods allow the user to rapidly carry out multiple immunoassays in the same sample volume, and to rapidly resolve the results of those immunoassays in an electronically assisted format. The assays may be further multiplexed in that several samples may be analyzed and visualized on the same microelectronic array. In addition, the methods and procedures of the invention allow the use of electronic stringency to further improve the specificity and accuracy of the immunoassays on the microelectronic array devices

This application is a continuation-in-part of Application Ser. No. 09/374,338, entitled “Microelectronic Molecular Descriptor Array Devices, Methods, Procedures, and Formats for Combinatorial Selection of Intermolecular Ligand Binding Structures and for Drug Screening,” filed Aug. 13, 1999, which is incorporated fully herein by reference.

### FIELD OF THE INVENTION

This invention relates to devices and methods for carrying out multi-step and multiplex immunoaffinity binding reactions in microscopic formats. In particular, these devices and methods allow the user to rapidly carry out multiple immunoassays in the same sample volume, and to rapidly resolve the results of those immunoassays in an electronically assisted format. In addition, The assays may be further multiplexed in that several samples may be analyzed and visualized on the same microelectronic array. In addition, the methods and procedures of the invention allow the use of electronic stringency to further improve the specificity and accuracy of the immunoassays on the microelectronic array devices.

### RELATED APPLICATIONS

This application is related to U.S. Pat. No. 6,051,380, entitled “Methods and Procedures for Molecular Biological Analysis and Diagnostics”, filed Dec. 5, 1997, U.S. Pat. No. 6,048,690, entitled “Methods for Electronic Perturbation Analysis of Biological Materials”, filed May 14, 1997, U.S. Pat. No. 5,849,486, entitled “Apparatus and Methods for Active Programmable Matrix Devices”, filed Sep. 27, 1995, U.S. Pat. No. 5,632,957, entitled “Molecular Biological Diagnostic Systems Including Electrodes”, filed Sep. 9, 1994, U.S. Pat. No. 6,017,696 entitled “Methods for Electronic Stringency Control for Molecular Biological Analysis and Diagnostics”, filed Jul. 7, 1994, and U.S. Pat. No. 5,665,662, entitled “Active Programmable Electronic Devices for Molecular Biological Analysis and Diagnostics”, filed Nov. 1, 1993. This application is also related to Application PCT/US99/03080, entitled “Advanced Active Devices and Methods for Molecular Biological Analysis and Diagnostics”, filed Feb. 11, 1999. This application is also related to WO 97/43232, entitled “Novel Substance Library and Supramolecular Complexes Produced Therewith”, published Nov. 20, 1997, WO 99/15539; WO 99/15539, entitled “Pentopyranosyl Nucleoside, and Production and Use of the Same”, filed Sep. 22, 1997; WO 99/15541, entitled “Pentopyranosyl Nucleoside for Producing an Electronic Component, and Conjugates of Said Pentopyranosyl Nucleoside”, filed Sep. 22, 1997; WO 99/15509, entitled “Cyclohexyl and Heterocyclyl Nucleoside Derivatives, Method for Producing These Derivatives, and the Use of the Derivatives and Their Oligomers or Conjugates in Pairing and/or Testing Systems, filed Sep. 22, 1997; WO 99/15542, entitled “Linker Nucleoside, and Production and Use of the Same”, filed Sep. 22, 1997; WO 99/15893, entitled “Addressable Modular Recognition System, Production Mode and Use”, filed Sep. 22, 1997; WO 98/25943, entitled “Non-Helical Supramolecular Nanosystems”, filed Dec. 11, 1996. The foregoing are hereby incorporated by reference as if fully set forth herein.

### BACKGROUND OF THE INVENTION

Immunoassays are a critical tools in the practice of in vitro immunodiagnostics, particularly in the detection and quantification of proteins such as those associated with cancer, liver disease or myocardial infarctions. Immunoassays also are becoming increasingly important in the field of proteomics, particularly in the detection of proteins and in the determination of protein-protein interactions. In any of these applications, it is often desirable to perform two or more different assays on the same sample, in a single device, and preferably at about the same time. Such assays are known in the art as multiplex assays. Multiplex assays are performed to determine simultaneously the presence or concentration of more than one molecule in the sample being analyzed, or alternatively, to evaluate several characteristics of a single molecule, such as, the presence of several epitopes on a single protein molecule. Simultaneous, discrete analysis of multiple analytes is employed in so called panel testing and screening assays. In panel testing, for a given specimen of interest several defined assays are ordered together for the purpose of reaching a diagnosis. In screening assays, the same assay or group of assays is performed on every specimen to determine the presence or concentrations of one or more of the analytes in the screen. In either of these situations an assay technology that affords the simultaneous discrete analysis of multiple analytes in a single device would have significant cost and convenience benefits.

One common type of immunoassay is the so called solid phase assay. Solid phase assays are characterized by the presence of a binding element, typically an antibody, immobilized on a solid support. Solid phase assays have been used to determine the presence and/or the concentration of biomolecules, such as proteins, peptides, nucleic acids, carbohydrates and lipids. Solid-phase assays can be performed in a variety of fluids, e.g., simple buffers, biological fluids such as blood, plasma, serum, urine, saliva, or tissue homogenates, environmental samples, and many others. Immune reactions involving the formation of antibody-antigen complexes are exemplary of known chemical or biochemical analyte binding reactions in which a complex is formed by the highly specific binding of the reaction moieties to one another. A number of other such reactions are known, for example, nucleic acid hybridizations, enzyme-inhibitor, enzyme-coenzyme, hormone-receptor, enzyme-receptor and like substrate-specific reactions.

Assays based upon these well known immune and other specific binding reactions involve a wide variety of techniques. Some assay methods employ radioactive, luminescent or fluorescent tags that are coupled to either the binding molecule or to the analyte, and can be detected by measuring radiation arising from the reaction product or complex. In the so called competitive assay configuration, a mixture of the analyte and a known amount of labeled analyte are applied to the immobilized phase. These compete with each other for the binding sites on the immobilized binding molecule. The greater the amount of the sample analyte present, the less will be the extent to which labeled analyte binds to the binding element. Using predetermined calibration curves and measuring the intensity of the signal obtained from the labeled antigen complexed with the solid-phase binding element, one can determine or assay the amount of unlabeled or sample analyte.

In another common technique is the direct, or sandwich, assay configuration. This assay type is often employed when analytes, typically proteins, have multiple epitopes or discreet binding sites that are sufficiently well separated spatially to allow two antibodies, or other binding elements, to bind simultaneously or sequentially. Typically, the sample is first incubated with the so called capture antibody, which reacts with the first epitope on the protein. This capture antibody may be pre-immobilized on a solid support, or may become immobilized subsequent to the analyte binding reaction (e.g., by a streptavidin-biotin interaction). The solid phase is then washed to remove unreacted components, and then further incubated with a labeled detecting 2^{nd} antibody, which binds to the 1^{st}-antibody-antigen complex. Unreacted excess 2^{nd} antibody is them removed by washing and the activity bound to the solid phase is determined. The greater the amount of the sample analyte present, the more labeled 2^{nd} antibody will be detected. Using predetermined calibration curves and measuring the intensity of the signal obtained, one can determine the amount of unlabeled sample analyte.

Multiplex assaying, especially on the large scale of proteomic applications, which require hundreds or thousands of assays, presents a significant challenge. Small scale multiplexing can be done with multiple labels and multi-wavelength detection. But the practical limit of this approach is set by the availability of discrete wavelength labels with adequate spectral separation. Another approach is to use physical separation of the assay sites, such as a two-dimensional array of binding sites. This approach is particularly useful in a miniaturized form often referred to as the microarray or biochip. The biochip format is compatible with many advanced fabrication methods and is well suited to automated manufacture and processing. The three primary technologies presently used in microarray manufacture are photolithography, ink-jetting, and various forms of mechanical microspotting. Of these only ink-jetting and mechanical spotting are practical for the construction of antibody arrays. Regardless, of their specific method of construction, all of these so called passive arrays suffer from the limitation that, once constructed, all sites on the array must be subjected to the same chemical reaction conditions. There is no practical way to vary conditions at each individual binding site. Such passive arrays are also limited in that the binding of any analytes or additional binding elements is facilitated only by diffusion, and is therefore a slow process.

The use of natural or biological nucleic acids to specifically attach a suite of peptide-sequence-epitope specific antibodies to an array has been proposed in theory for the purpose of protein or peptide sequencing analysis, see U.S. Pat. No. 5,800,992, column 8, lines 29-50. However, this approach is not practicable utilizing conventional nucleic acid techniques. While many DNA array technologies are available and could be applied to this type of processes, their limited stringency parameters may present problems with obtaining high level selectivity for the positioning of the antibodies (see A. Marshall and J. Hodgson, Nature Biotechnology, Vol. 16, pp. 27-31, 1998, and G. Ramsay Nature Biotechnology, Vol. 16, pp. 40-44, 1998), and the thermodynamic limitations of passive hybridization techniques necessitate long incubation times on the order of tens of hours.

Problems with sensitivity and specificity have so far limited the applications of nucleic acid hybridization. Nucleic acid hybridization analysis generally involves the detection of a very small numbers of specific target nucleic acids (DNA or RNA) with probes among a large amount of non-target nucleic acids. In order to keep high specificity, hybridization is normally carried out under the most stringent condition, achieved through a combination of temperature, salts, detergents, solvents, chaotropic agents, and denaturants. Multiple sample nucleic acid hybridization analysis has been conducted on a variety of filter and solid support formats (see G. A. Beltz et al., in Methods in Enzymology, Vol. 100, Part B, R. Wu, L. Grossmam, K. Moldave, Eds., Academic Press, New York, Chapter 19, pp. 266 308, 1985). One format, the so-called “dot blot” hybridization involves the attachment of target DNAs to a filter, which are subsequently hybridized with a labeled probe(s). “Dot blot” hybridization gained wide-spread use, and many versions were developed (see M. L. M. Anderson and B. D. Young, in Nucleic Acid Hybridization—A Practical Approach, B. D. Hames and S. J. Higgins, Eds., IRL Press, Washington D.C., Chapter 4, pp. 73 111, 1985).

Several problems which have plagued the use of nucleic acid probes for the study of genomics and gene expression also pose problems for the use of nucleic acid oligomers as addressing reagents. One problem relates to the stringency control of hybridization reactions. Hybridization reactions are usually carried out under the stringent conditions in order to achieve hybridization specificity. Methods of stringency control involve primarily the optimization of temperature, ionic strength, and denaturants in hybridization and subsequent washing procedures. Unfortunately, the application of these stringency conditions causes a significant decrease in the number of hybridized probe/target complexes for detection.

Another problem relates to the high complexity of DNA in most samples, particularly in human genomic DNA samples. When a significant number (more than 5 or so) pairs of nucleic acids are used for addressing molecules, similar complexity problems are present. When a sample is composed of a number of sequences that are closely related to the specific target sequence, even the most unique probe sequence has a large number of partial hybridizations with non-target sequences. A third problem relates to the unfavorable hybridization dynamics between a probe and its specific target. Even under the best conditions, most hybridization reactions are conducted with relatively low concentrations of probes and target molecules. In addition, a probe often has to compete with the target strand's complementary nucleic acid for hybridization with the target sequence. A fourth problem for most present hybridization formats is the high level of non-specific background signal. This is caused by the affinity of DNA probes to almost any material. These problems, either individually or in combination, lead to a loss of sensitivity and/or specificity for nucleic acid hybridization in the described formats.

New techniques are being developed for carrying out multiple nucleic acid hybridization analysis on micro-formatted multiplex or matrix devices (e.g., DNA chips and arrays) (see M. Barinaga, 253 Science, pp. 1489, 1991; W. Bains, 10 Bio/Technology, pp. 757-758, 1992). These methods usually attach specific DNA sequences to very small specific areas of a solid support, such as micro-wells of a DNA chip. These hybridization formats are micro-scale versions of the conventional “dot blot” and “sandwich” hybridization systems. However, the actual mechanism by which the DNA probes find their complements is only changed in that the sample hybridization volume is decreased: the other variables related to hybridization are the same as for traditionally scaled “dot blot” reactions.

Fodor et al., 251 Science 767-773, 1991, used photolithographic techniques to synthesize oligonucleotides on a glass matrix. Pirrung et al., in U.S. Pat. No. 5,143,854, Sep. 1, 1992, teach large scale photographic solid phase synthesis of polypeptides in an array fashion on silicon substrates. Fodor et al., 364 Nature, pp. 555-556, 1993, used an array of 1,024 8-mer oligonucleotides on a solid support to sequence DNA. In this case, the target DNA was a fluorescently labeled single-stranded 12-mer oligonucleotide containing only nucleotides A and C. 1 pmol (˜6×1011 molecules) of the 12-mer target sequence was necessary for the hybridization with the 8-mer oligomers on the array. The results showed many mismatches. Like Southern, Fodor et al. did not address the underlying problems of direct probe hybridization, such as stringency control for multiplex hybridizations. In order to achieve optimal stringency and selectivity for target DNA sequences a large number of immobilized capture probe controls must be incorporated into the array in order to assess the degree of mismatch hybridization under the reaction conditions (usually 20 or more for determining single base mismatches, see M. J. Kozal et al., Nature Medicine, Vol. 2, No. 7, pp. 753-759, 1996.) See, e.g., U.S. Pat. No. 5,744,305, Fodor et al., entitled “Arrays of Materials Attached to a Substrate”.

Drmanac et al., 260 Science 1649-1652, 1993, used the above discussed second format to sequence several short (116 bp) DNA sequences. Target DNAs were attached to membrane supports (“dot blot” format). Each filter was sequentially hybridized with 272 labeled 10-mer and 11-mer oligonucleotides. A wide range of stringency condition was used to achieve specific hybridization for each n-mer probe; washing times varied from 5 minutes to overnight, and temperatures from 0° C. to 16° C. Most probes required 3 hours of washing at 16° C. The filters had to be exposed for 2 to 18 hours in order to detect hybridization signals. The overall false positive hybridization rate was 5% in spite of the simple target sequences, the reduced set of oligomer probes, and the use of the most stringent conditions available. See, e.g., U.S. Pat. No. 5,695,940, Drmanac et al, entitled “Method of Sequencing by Hybridization of Oligonucleotide Probes”.

Regardless of the format, current micro-scale DNA hybridizations systems and passive DNA chip/array approaches do not overcome the underlying problems associated with nucleic acid hybridization reactions. They require very high levels of relatively short single-stranded target sequences or PCR amplified DNA, and can still produce a high level of false positive hybridization signals even under the most stringent conditions. In the case of multiplex formats using passive arrays of short oligonucleotide sequences, it is not possible to optimize the bulk stringency condition for each individual sequence with any conventional approach because the arrays or devices used for these formats can not change or adjust the temperature, ionic strength or denaturants at an individual test sites or locations relative to other locations. Therefore, a common stringency condition must be used for all the sequences on the device. This results in a large number of non-specific and partial hybridizations and limits the application of the passive hybridization arrays and devices. The problem becomes more compounded as the complexity and number of different target sequences on the array increases.

Thus, the use of passive array hybridization systems as addressing components in an antibody arraying strategy is not very practicable: the very passive nature of these DNA array technologies presents a problem. These passive arrays are unable to independently provide the diversity of stringency conditions that are needed to accurately address antibodies utilizing reasonable antibody concentrations in a timely manner. Using passive array technology with limited bulk stringency parameters (temperature, pH, ionic strength, etc.) would require inordinate amounts of time to carry out the addressing of an array of antibodies for “peptide sequencing,” as suggested by Fodor. Secondly, such arrays offer no advantages over more traditional mechanical-spotting techniques, which can be used to place several dozen to several hundred antibodies within a square centimeter. Both traditional antibody arrays and the Fodor proposed array are only useful for mass screening of a single sample against a set of previously immobilized antibodies, and cannot be used to resolve multiplex immunological reactions within a span of time comparable to traditional microtiter well ELISA-type assays.

Clearly then, there is a need in the practice of immunodiagnostics and proteomics, to be able to assemble rapidly and specifically arrays of antibodies or other binding elements, to provide means of accelerating binding reactions otherwise limited by diffusion, and to be able to control independently and specifically the reaction conditions at the individual sites on the array.

### SUMMARY OF THE INVENTION

The present invention overcomes the limitations and impracticalities of these passive array constructs by utilizing active matrices with individually controlled test sites. The individually controlled test sites of the active electronic matrices utilized in the invention allow the conditions at each individual test site to be varied, altering the pairing of the pairing component member attached to an immunoreaction component (IₙC_{B}-Pₓ) with its complement pairing component member (Pₓ′) attached to the test site. This allows for the rapid, selective pairing of each IₙC_{B}-Pₓ with its Pₓ′ when the individual test site is biased with an electric charge, providing the means to resolve in minutes complex immunoreactions involving several antibody-antigen interactions in the same sample volume. Because the individual test sites may be oppositely biased, or unbiased, a second test site with the same Pₓ′ can be utilized on the same active matrix surface, without binding of the IC-Pₓ to the second test site. This selective addressing ability allows different IC's, or entire immunoreaction complexes, with the same Pₓ's to be addressed to different sites. Thus, the same pairing components may be utilized to rapidly and selectively address different IC's to different test sites, or to resolve multiple sample immunoreactions on different sets of test sites, all on the same active matrix surface. In addition, the electric fields produced by the active matrix may be utilized to concentrate charged immunoreactants at the test sites (electronic incubation), providing decreased reaction time and selective addressing of sample immunoreactants.

Thus, in a primary aspect, this invention relates to methods for the use of active microelectronic array devices to rapidly carry out multiplexed immunological reactions. The immunological reactions may be multiplex both in terms of resolving multiple antigen-antibody reactions occurring in the same sample volume, and in terms of selectively reacting or resolving multiple samples on the same active matrix surface. In another aspect, this invention relates to novel compositions of matter which are formed in preparation for, and through the action of, these methods, including active electronic matrix devices which comprise immunological reaction components (IₙC's) and pairing components (Pₓ's) in various configurations.

In a first group of embodiments of the immunoreaction methods of the invention, the active electronic matrix is used to quickly address one or more immunoreaction complexes to a test site in the active matrix, “resolving” the products of the immunoreaction, and thereafter detecting the presence of the immunoreaction complex at the test site. Thus, the first step in these methods is contacting a sample which may contain at least a first analyte immunoreaction component (I₁C_{A}) with at least a first binding immunoreaction component-first pairing component member complex (I₁C_{B}-P₁), thereby producing an immunoreaction complex (I₁C_{A}-I₁C_{B}-P₁) if the analyte is present. The immunoreaction mixture is then contacted with an active electronic matrix, comprising a plurality of test sites, wherein a complementary first pairing component member (P₁′) is attached to at least one test site, and wherein the test site is electrically biased to promote the pairing of the members of the first pairing component. The pairing component members then selectively pair, creating an addressed immunoreaction complex, I₁C_{A}-I₁C_{B}-P1-P₁′, attached to the test site. The immunoreaction complex may then be detected at the test site, whereby the presence of the first analyte immunoreaction component in the sample may be determined. Detection is usually achieved by incorporating a labeled immunoreaction (I₁C_{L}) component which binds to the analyte immunoreaction component (I₁C_{A}) in the immunoreaction complex. This detectable immunoreaction component may be added to the sample immunoreaction mixture and addressed as a part of the immunoreaction complex, or may be later incubated with the active matrix in order to bind to the addressed immunoreaction complexes. Alternatively, competitive immunoassay formats may be used, in which a known amount of a labeled analyte standard (I₁C_{A}*) is added to the immunoreaction mixture. The presence and quantity of the unlabeled antigen in the sample may then be determined by the decrease in the incorporation of the labeled analyte standard into the addressed immunoreaction complexes.

These methods may be multiplexed in two ways. The first is by simultaneously forming multiple immunoreaction complexes with multiple analyte immunoreaction components in the sample immunoreaction mixture, and then resolving the -immunoreaction complexes onto the test sites of the active electronic matrix. In these embodiments, the sample which may contain at least a first analyte immunoreaction component (I₁C_{A}) and a second analyte immunoreaction component (I₂C_{A}) is combined with at least a first and second binding immunoreaction component- pairing component complex (I₁C_{B}-P₁ and I₂C_{B}-P₂), thereby producing immunoreaction complexes (I₁C_{A}-I₁C_{B}-P₁ and I₂C_{A}-I₂C_{B}-P₂) if the analytes are present. The immunoreaction mixture is then contacted with an active electronic matrix, comprising a plurality of test sites, wherein a complementary first pairing component member (P₁′) is attached to at least one first test site, and a complementary second pairing component member (P₂′) is attached to at least one second test site, wherein the first and second test sites are electrically biased to promote the pairing of the members of the first and second pairing components. The pairing component members then selectively pair, creating a first addressed immunoreaction complex, I₁C_{A}-I₁C_{B}-P₁-P₁′, attached to the first test site, and a second addressed immunoreaction complex, I₂C_{A}-I₂C_{B}-P₂-P₂′, attached to the second test site. The addressed immunoreaction complexed may then be detected at the test sites. In analogous embodiments, three, four, five, and even ten or more, analyte immunoreaction components may be resolved and detected in this manner.

In addition, these methods may be multiplexed by the selective addressing of different sample immunoreaction mixtures to different test sites, or sets of test sites. For illustration, in the above double multiplexed immunoreaction, the two immunoreaction complexes I₁C_{A}-I₁C_{B}P₁ and I₂C_{A}-I₂C_{B}-P₂ from a first sample immunoreaction may be selectively addressed to test sites 1A and 1B in a first row of the active electronic matrix. Then, a second immunoreaction mixture may be resolved by biasing test sites 2A and 2B in a second row, also containing P₁′ and P₂′, thus attaching the second set of immunoreaction complexes to 2A and 2B . The presence of the analyte immunoreaction components in each sample may then be detected by, for instance, incubating the entire active electronic matrix surface with labeled immunoreaction components I₁C_{L} and I₂C_{L}, thus labeling both the IR₁ and IR₂ complexes. Because the individually controlled test sites may be made highly selective for the hybridization or binding of the pairing components, three, four, five, ten, or several dozen samples may be analyzed in this manner, and compared side by side on the active matrix.

In other embodiments of the immunoassay methods of the invention, the binding immunoreaction components are first addressed to test sites on the active electronic matrix, and then reacted with analyte immunoreaction components in the sample. Thus, in their simplest form, the first step in these methods is to contact at least a first binding immunoreaction component-first pairing component member complex (I₁C_{B}-P₁) with an active electronic matrix comprising a plurality of test sites, wherein a complementary first pairing component member (P₁′) is attached to at least one test site, and wherein the test site is electrically biased to promote the pairing of the members of the first pairing component. The pairing component members then selectively pair, creating an addressed binding immunoreaction component complex, I₁C_{B}-P₁-P₁′, attached to the test site. The active electronic matrix is then incubated, either electronically or passively, with a sample which may contain an analyte immunoreaction component I₁C_{A}, thus forming an attached immunoreaction complex I₁C_{A}-I₁C_{B}P₁-P₁′. The attached complex may then be detected at the test site.

Even in this simplest form, and when the sample is passively incubated with the matrix, the immunoassay of the invention realizes a significant advantage over passive hybridization array technologies in that the amount of time needed to hybridize a given concentration of the I₁C_{B}-P₁ complex to the P₁′ attached to the test site is greatly reduced, thus allowing the use of very dilute I₁C_{B}-P₁ complex concentrations. However, even greater advantages are realized by utilizing the methods of the invention to electronically address at least two sets of immunoreaction components to at least two sets of test sites with the same set of pairing components. To illustrate these methods, a first set of immunoreaction component-pairing component complexes, I₁C_{B}-P₁ and I₂C_{B}-P₂, may be selectively addressed to test sites 1A and 1B containing P₁′ and P₂′, in a first row of the active electronic matrix. Then, a second set of immunoreaction component-pairing component complexes, I₃C_{B}-P₁ and I₄C_{B}-P₂, may be addressed by biasing test sites 2A and 2B , containing P₁′ and P₂′, in a second row on the same active electronic matrix. Thus, by utilizing the selective activation of sets of test sites in two rounds, a set of two pairing components may be utilized to address four binding immunoreaction components to four test sites. Analogously, a set of ten pairing components may be utilized to address 100 different immunoreaction components to 100 test sites in ten rounds of electronic addressing.

In addition to accelerated and selective pairing component addressing of binding immunoreaction component-pairing component complexes, the analyte immunological components may also be electronically addressed to the test sites in the methods of the invention, depending on the charge characteristics of the analyte. In these embodiments of the methods of the invention, the binding immunoreaction component-pairing component complexes are first electronically addressed to a set of test sites in the active electronic matrix, as described above. Then, the sample is electronically incubated with the matrix by electronically biasing the test sites so as to concentrate any analyte immunoreaction components present in the sample at the activated test sites. The analytes in the sample rapidly react with the binding immunoreaction components, forming immobilized immunoreaction complexes. These may then be detected by any of the means described above.

In addition to accelerated reaction, the electronic incubation methods used in the immunoassays of the invention also allow the selective addressing of sample analyte immunoreaction components to the test sites of the active electronic matrix. To illustrate these methods, a set of immunoreaction component-pairing component complexes, I₁C_{B}-P₁ and I₂C_{B}-P₂, may be selectively addressed to test sites 1A and 1B , containing P₁′ and P₂′, in a first row of the active electronic matrix, and to test sites 2A and 2B , also containing P₁′ and P₂′, in a second row of the active electronic matrix. A first sample is then contacted with the matrix while test sites 1A and 1B are appropriately electronically biased, selectively concentrating and reacting the analytes I₁C_{A} and I₂C_{A} at those test sites. A second sample may then be contacted with the matrix while test sites 2A and 2B are appropriately biased, selectively concentrating and reacting the analytes I₁C_{A} and I₂C_{A} from the second sample at those test sites. In this fashion, several samples may be analyzed utilizing selected portions of the same active electronic matrix with negligible or insignificant side reactions in the unselected portions of the matrix, even though the unselected portions contain the same attached binding immunoreaction components.

As is evident from the above summary of the methods of the invention, several novel compositions of matter are created in preparation for, and during, the methods in order to detect the analyte immunoreaction component in the sample. In a simplified single-site version, the composition comprises an attachment surface in an active electronic matrix test site, the attachment surface thus being located in a controlled electronically variable environment. Attached to the surface of the test site is a first pairing component (P₁ and P₁′), wherein at least a first member (P₁′) of the first pairing component is attached to the surface, and the complementary member (P₁) is paired with P₁′. A binding immunological reaction component (I₁C_{B}), which may be an antibody, antibody fragment, antibody derivative, synthetic antibody, or a molecular species bearing an immunologically reactive epitope (antigen), is attached to P₁. In a further embodiment, an antigen or antibody of interest, or the analyte immunoreaction component (I₁C_{A}) may be attached to I₁C_{B} to form an immunoreaction complex. In further embodiments, other immunological reaction components, such as labeled immunoreaction components (I₁C_{L1}, I₁C_{L2}, I₁C_{L3} . . . ), may be bound to I₁C_{A} to form immunoassay-sandwich complex layers for the purposes of detecting the presence of the I₁C_{A}. Most preferably, the pairing component pair components are complementary and coded pairing components with uniform physio-chemical characteristics, such as p-RNA, other synthetic nucleic acid-like molecules, and nucleic acids.

In a more complex multisite version, the combinations of matter of the invention comprise an active electronic matrix with a first test site as described above, and at least one second test site, wherein the second test site also contains a first member (P₁′) of the first pairing component attached to the surface. However, the amount of I₁C_{B}-P₁, and any other attached immunoreaction complex components, which is attached via the first pairing component member to the first test site is significantly greater than the amount of I₁C_{B}-P₁ attached to the second test site, and wherein the first test site and second test site were connected with a suitable aqueous liquid when the members of the pairing component were allowed to pair. Such a composition is formed by selective biasing of the test sites to electronically address the I₁C_{B}-P₁ complex. In a further embodiment, the second test site has a significantly greater amount of a second binding immunoreaction component-first pairing component complex (I₂C_{B}-P₁) attached to the second test site than to the first test site, wherein the first test site and second test site were connected with a suitable aqueous liquid when the members of the pairing component were allowed to pair.

In another complex multisite version, the combinations of matter of the invention comprise an active electronic matrix with a first test site with I₁C_{B} attached via a pairing component, and at least one second test site with I₁C_{B} attached via a pairing component wherein the first and second pairing components may be the same, or different. The composition further comprises an analyte immunoreaction component I₁C_{A}¹, derived from a first sample, which is attached to I₁C_{B} at the first test site in amounts significantly greater than that attached to I₁C_{B} at the second test site, wherein the first test site and second test site were connected with a suitable aqueous liquid when the immunoreaction was allowed to occur. This composition is formed when the sample is selectively addressed and electronically incubated with the first test site by differently biasing the first test site as compared to the second test site. these compositions may also further comprise labeling immunoreaction components for the detection of the analyte.

Another complex multisite composition comprises an active electronic matrix with a first test site with I₁C_{A}¹-I₁C_{B}-P₁-P₁′ attached to the test site, wherein the analyte immunoreaction component is derived from a first sample. The composition further comprises at least one second test site with I₁C_{A}²-I₁C_{B}-P₁-P₁′ attached to the second test site, wherein the analyte immunoreaction component is derived from a second sample, wherein the amount Of I₁C_{A}¹-I₁C_{B}-P₁-P₁′ attached to the first test site is significantly greater than the amount attached to the second test site, and wherein the amount of I₁C_{A}²-I₁C_{B}-P₁-P₁′ attached to the second test site is significantly greater than the amount attached to the first test site. Further, the first test site and second test site were connected with a suitable aqueous liquid when the pairing of the pairing components was allowed to occur. This composition is formed when immunoreaction complexes are separately electronically addressed to the test sites from first and second sample immunoreaction mixtures. These compositions may also further comprise labeling immunoreaction components for the detection of the analyte.

In another aspect, the present invention provides kits for performing the above assay methods on an active electronic matrix device. In one embodiment, these kits comprise a set of binding immunoreaction component-pairing component member complexes (I₁C_{B}-P₁, . . . , IₙC_{B}-Pₓ), for each analyte (I₁C_{A}, . . . , IₙC_{A}) to be detected. In this embodiment, the kits may also contain functionalized complementary pairing component members (P₁′-Pₓ′) for attachment to the test sites of the device. In another embodiment, the kit may contain sets of pairing component members which may be functionalized for attachment to the test sites of the active electronic matrix device and/or for attachment to various IₙC_{B}. In addition, the kits may contain components independently selected from: buffers, labeled analyte standards, labeled immunoreaction components, instructions for their use in the methods of the invention, or other components useful for particular research or clinical applications of the methods of the invention.

### BRIEF DESCRIPTION OF THE DRAWINGS

FIG. 1: An illustration showing the structural differences, both on a monomeric level and on a secondary structure level, between paired deoxyribonucleic acid and paired pyranosyl-ribonucleic acid, p-RNA. Note that the sugar moiety in the p-RNA monomer contains a six-membered ring, rather than the five-membered ring of a deoxyribonucleotide. This conveys a planar secondary structure on p-RNA, rather than the three-dimensional helical structure of nucleic acids such as DNA.

FIG. 2: A schematic of the synthesis of the β-D-ribopyranosylphosphoramidite of tryptamine from phthalyl-N-tryptamine. The reaction conditions at each step are as follows: i) 1M-borane-THF, CF₃CO₂H, 0° C., 30 min; ii) D-ribose, dry ethanol, reflux, 4h; iii) Ac₂O, py., rt, 18h; iv) DDQ, CH₂Cl₂, rt, 1.5h; v) MeONa, dry methanol, rt, 18h; vi) C₆H₅COCl, CH₂Cl₂, DMAP, py., −78° C., 15 min; vii) DMTCl, CH₂Cl₂, DMAP, py. DIPEA, molecular sieves, rt, 4.25h; viii) DMAP, py., DIPEA, n-propanol, p-nitrophenol, 75-80° C., 96h; ix) ClP(Oallyl)(iPr₂N), DIPEA, CH₂Cl₂, rt, 2h.

FIG. 3: A diagram of an IgG immunoglobulin, showing the ficin cleavage site. The brick-patterned sections indicate the variable regions of the heavy and light chains, and the cross-hatched sections indicate the constant regions. Cysteines forming disulfide linkages are indicated by gray rectangles.

FIG. 4a & 4b: An illustration of active matrix electronic addressing of binding immunoreaction component-pairing component member complexes to complementary pairing components on the activated test sites. Both antibody (Y structure) and antigen (blob structure) binding immunoreaction components are illustrated. different shading/patterns indicate different immunoreaction components. Note that six different immunoreaction components are addressed to six different sites utilizing three pairing components in the illustration.

FIG. 5: An illustration of resolving a complex sample immunoreaction mixture utilizing the active matrix methods of the invention. In this illustration, labeling immunoreaction components (antibodies with attached fluorescent moieties [sunbursts]) are added to the immunoreaction mixture, and the entire labeled immunoreaction complex is electronically addressed to the test sites according to the pairing components. Alternatively, the labeling immunoreaction components may be added after electronically addressing of the analyte-binding immunoreaction component complex to the test sites of the matrix.

FIG. 6: An illustration of the resolution of a second complex sample immunoreaction mixture to an active electronic matrix, after a first sample immunoreaction mixture been resolved on the same matrix. Note that the incidence of the specific interactions of the pairing components is dramatically increased at the activated test sites, due to the electric field generated by the positively biases electrode. Thus, the interaction of pairing component members at the non-biased test sites is negligible compared to the biased test sites. This allows for the resolution of multiple samples on the same matrix, without cross-reactions between the samples and un-biased rows or sets of test sites.

FIG. 7a & 7b: An illustration of the electronic addressing of binding immunoreaction components to the active matrix, and subsequent incubation of sample analytes with the matrix. In this illustration, the analytes are negatively charged, and thus the sample is electronically incubated with the binding immunoreaction components attached to the matrix.

FIG. 8a & 8b: Photomicrographs of the microelectronic array, showing the results of the experiment in Example 5. FIG. 8a was taken using a Cy3 filter, and FIG. 8b was taken using a Cy5 filter.

FIG. 9: A Biacore Sensogram showing the p-RNA pairing component hybridization results of the experiment in Example 6.

### DETAILED DESCRIPTION

The invention relates to the design and fabrication of addressable active microelectronic array devices, and the processes, procedures, techniques, formats, methods and uses of these devices to carry out multi-step and multiplex immunoreactions in microscopic formats. These devices and methods allow for several different immunoassay formats and compositions, which all rely on the use of pairing components and to resolve these immunoreactions to discrete locations on the active matrix devices.

The principles of the invention are illustrated with the use of electronically controlled arrays, in which electronic fields are utilized to selectively address the binding immunoreactant component-pairing component member complexes IₙC_{B}-Pₓ) to their complementary pairing component members (Pₓ′) attached to the test site of the active electronic matrix. However, the use of devices other than the APEX devices described in the below referenced patents are also contemplated by the present invention. For instance, the methods and compositions of the invention may be readily adapted for use with the marcrohybridization devices described in copending U.S. Ser. No. 09/671,594, filed Sep. 27, 2000, entitled “Electronic Systems, Component Devices, Mechanisms, Methods, and Procedures for Macroscopic and Microscopic Molecular biological Reactions, Analyses, and Diagnostics.” The devices described therein are also considered to be active electronic matrices, as the term is used herein. The use of the term test site in the present application also encompasses the term “test site microlocation” as defined in that application, which is incorporated fully herein by reference.

In addition, the use of similar types of devices which operate on principles other than electric fields to activate the test sites of the matrix are also contemplated for use in the methods of the present invention. Such devices would have some means of remotely activating each test site of the active matrix individually (i.e., not simply by mechanical spotting onto the matrix surface). Examples of such activation strategies include the use of radiation to remotely alter the test site characteristics during the methods, or the use of micro-capillaries or electrochemistry to generate an altered chemical environment at the test site during the methods. However, electric fields are the preferred means of altering the test site environment, as they provide a means for concentrating relatively dilute reactants at the test sites, thus increasing the rate of binding reactions at those sites.

In general, the active electronic matrices utilized in the present invention consist of a planar substrate comprising an array of independently (individually) or semi-independently (in sets or groups) controlled electrodes. The array may be in any convenient geometric arrangement, including lines, radially symmetrical patterns, rectilinear grids, etc. One or more electrodes may be differently sized than the other electrodes in the array, and/or differently placed, in order to serve as a reference electrode or storage portion of the matrix device. The substrate is covered by a permeation layer, which may be contiguous on the substrate, but which is at least above the electrodes of the array. Alternately, the permeation layer may be separated from the electrode by a buffer reservoir. This permeation layer is permeable to small ions, but protects the biomolecular reactants from the harsh electrochemical environment of the electrode. The area of the permeation layer above an electrode in the array forms a “test site.” The permeation layer contains, at least on its surface at each test site, reactive or binding moieties which allow the attachment of the first member of the component pairs. Thus, the active electronic matrix forms the base for the compositions and methods of the invention.

Active microelectronic chip/array technologies have been demonstrated which provide capability for selectively addressing arrays with nucleic acid sequences, carrying out rapid multiplex hybridization, and also providing electronic stringency for improving nucleic acid hybridization selectivity. These same basic microelectronic arrays can be used for the immunological reaction methods and compositions of matter that are the subject of this invention. The basic designs and procedures for fabricating microelectronic DNA chips and arrays, particularly higher density devices (10,000 active sites), are described, for example, in U.S. Pat. No.: 6,017,696, entitled “Methods for Electronic Stringency Control for Molecular Biological Analysis and Diagnosis;” U.S. Pat. No. 5,605,662, entitled “Active Programmable Electronic Devices for Molecular Biological Analysis and Diagnostics;” and U.S. Pat. No. 6,099,803, entitled “Advanced Active Electronic Devices for Molecular Biological Analysis and Diagnostics,” U.S. Pat. No. 5,632,957, entitled “Molecular Diagnostic Systems Including Electrodes;” and U.S. Pat. No. 5,849,486, entitled “Apparatus and Methods for Active Programmable Matrix Devices;” each of which is incorporated fully by reference herein.

In particular, those electronic stringency parameters (DC, AC/DC, and electronic pulsing protocols) which concern electronic perturbation, have been described in patents and applications which deal with the so-called area of fluorescent perturbation. See, e.g., U.S. Pat. No. 5,849,486, entitled “Apparatus and Methods for Active Programmable Matrix Devices.” Additionally, the design and fabrication procedures for higher density microelectronic arrays (e.g., 400, 1200, 10,000, and higher numbers of test sites) that have active on-board electronic control have been described in the above patents. Further information on the basics of electronic hybridization and stringency are also discussed in the following reference articles (Heller, M. J., IEEE Engineering in Medicine and Biology, pp. 100-104, March/April 1996; Sosnowski, R., et al., Proc. Nat. Acad. Sci. 94, pp. 1119-1123, 1997; Edman, C. F., et al., Nucleic Acid Research 25, pp. 4907-4914, 1997; Cheng, J., et al., Nature/Biotechnology 16, pp. 541-546, June 1998; and Gilles, P. N., et al., Nature/Biotechnology 17, No. 4, pp. 365-370, 1999).

The various electronic methods, procedures and formats for carrying out electronic addressing of the arrays, active electronic hybridization and electronic stringency all serve as a basis for the addressing and programmable pairing hybridization reactions described in the invention. However, the methods of the invention go beyond the teachings of this prior work in showing that, unexpectedly, the charged pairing components (Pₓ's) attached to the binding immunoreaction components (IₙC_{B}'s) of the inventions can be utilized to transport and selectively locate both individual IₙC_{B}'s as well as whole immunoreaction complexes (antibody-antigen-antibody sandwiches, and the like), and that the active electronic matrices may further often be utilized to selectively electronically concentrate and incubate sample antigens or antigen-antibody complexes under conditions which allow immunoreaction with immobilized IC's on individual matrix test sites. This level of demonstrated electronic control for a wide arrange of immunoreactions is remarkable, given the variability in the molecular bases for antibody-antigen interactions (hydrogen bonding, Van der Waal's interactions, and hydrophobic interactions), and the fundamental differences between nucleic acid hybridizations and these interactions.

It is to be understood that “attach” or “bind” as used in this application includes all covalent and non-covalent molecular interactions which produce a molecular association which is reasonably stable during the timescale of the methods of the invention. Such interactions include, for example, antibody-antigen interactions, nucleic acid hybridization interactions, streptavidin/avidin-biotin interactions, metal chelate interactions, protein binding pair interactions, protein/aptamer binding, and the like. In general, the attachments between immunoreaction components and between pairing component members will be non-covalent.

### Immunological Reaction Components

As used herein, “immunoreaction” or “immunological reaction” generally refers to a specific binding reaction between an antibody, or antibody-like molecule, and an antigen, or an epitope-bearing molecule, in addition to further specific binding interactions utilized to detect the antigen-antibody immunoreaction complex. In general, the immunological reactions will be described in terms of three types of components: binding components (C_{B}), analyte components (C_{A}), and labeling components (C_{L1}, C_{L2}, C_{L3}, . . . ). These are the basic components of most immobilized immunoassay formats, including traditional sandwich and competitive binding assay formats. In the case of sandwich formats, the immunoreaction complex may include one or more layers of labeling components in order to generate a signal to detect the presence of the analyte in the sample. In the case of competitive immunoassay formats, a known amount of the analyte itself is labeled, and the extent of the binding of this labeled analyte in the presence, and absence, of the sample is determined. It should be noted that C_{B} may be either an antigen or an antibody, depending on the analyte C_{A} to be detected. C_{L} usually comprises an antibody or antibody-like moiety and one or more detectable moieties. However, C_{L} also may be any other molecule which will attach to C_{A} with an affinity significantly greater than its affinity for the permeation layer, C_{B}, or other non-specific background material.

The immunoreaction components are described herein in terms of their position in relation to the test site surface. The first immunoreaction component, which is bound to a pairing component member, is the binding immunoreaction component C_{B}. Its purpose is to attach to, or capture, the analyte immunoreaction component C_{A} from the sample, and to link C_{A} to the test site location via the pairing component. C_{A} is simply the antigen or antibody which is the target of the immunoassay. Labeling components, C_{L}, if present, function to link the C_{A} to a detectable moiety. There may be several layers of labeling components, which may be utilized to amplify the detectable signal. These components are represented by a reference to their immunoreaction (e.g., I₁). Thus, an analyte immunoreaction component for a first immunoreaction is denoted I₁C_{A}, a binding immunoreaction component for the same immunoreaction is denoted I₁C_{B}, and so on.

For the purposes of this invention, an analyte or binding immunological reaction component is either 1) a molecule which presents an immunochemically reactive epitope or 2) a structure that has specific affinity for an epitope. The molecules of category 1 comprise those molecules generally called “antigens,” which present antigenic determinants which comprise a particular spatial arrangement of atoms which is recognizable by an antibody, or an “epitope.” Antigenic molecules which induce an immune response are usually quite large. Many antigens are proteins, polypeptides, polysaccharides, proteoglycans, glycoproteins, lippopolysaccharides, and other biologically derived macromolecules. Cells, bacteria, virus, cell surface membranes, cell surface proteins, cell surface receptor and effector sites, organelles, nuclei, mitochondria, ribosomes, synthetic micelles, and other natural or synthetic surfaces also present epitopes which can be recognized by antibodies, usually in the form of a molecular portion of the overall structure. However, the epitope recognized by an antibody is usually a small structure, and only a portion of the whole antigen. Thus, the molecules of category 1 also comprise smaller portions of an antigen which contain an epitope (e.g., periodate digested bacterial polysaccharides), as well as synthetic molecules (peptides, polysaccharides, etc.) which have been designed to mimic an epitope. For convenience, category 1 molecules will be referred to throughout as “antigens,” although this usage of the term understood to encompass all of the epitope-bearing molecules described above.

Generally, structures of category 2 include any multiple polypeptide chain-containing molecular structure that has a specific shape which fits to and recognizes an epitope, where one or more non-covalent binding interactions stabilize the complex between the molecular structure and the epitope. The specific or selective fit of a given structure and its specific epitope is sometimes referred to as a “lock and key” fit. The archetypal category 2 molecule is the antibody, and all types of immunoglobulins (IgG, IgM, IgA, IgE, IgD), immunoglobulin fragments comprising the binding site (i.e., Fab', papain, pepsin, or ficin fragments), derivatized immunoglobulins (with added chemical linkers, detectable moieties [fluorescent dyes, enzymes, substrates, chemiluminescent moieties], specific binding moieties [such as streptavidin, avidin, or biotin], etc.), recombinant immunoglobulins, single-stranded engineered immunoglobulins and humanized or hybrid immunoglobulins. Category 2 also may include artificial antibody-like molecules, such as the triad-peptide “finger” constructs described in copending U.S. Ser. No. 09/374,338, entitled “Microelectronic Molecular Descriptor Array Devices, Methods, Procedures, and Formats for Combinatorial Selection of Intermolecular Ligand Binding Structures and for Drug Screening,” filed Aug. 13, 1999, or analogs thereof. In addition, the category can include DNA or RNA oligomer-containing aptamers, metal chelators, and other non-proteinaceous specific binding molecules. For convenience, the term “antibody” will be used throughout to generally refer to category 2 molecules, although the term will encompass all immunoglobulins, derivatives, fragments, and modifications as described above.

The non-covalent interactions which bind the antibody and the antigen can include: hydrogen bonding, hydrophobic bonding, aromatic ring stacking, electrostatic interactions, chelation (with metal ion-containing epitopes) and van der Waals interactions. In addition to the physical/chemical parameters which determine epitope-antibody binding; the antibody binding sites may also have stereo-selective properties. In some cases, the same molecule or structure can serve both as an antibody and an antigen: an antibody molecule may serve as a receptor for a specific hapten molecule and also as a ligand for another antibody (usually of a different species.) This is often the case for antibody C_{A}'s, which may be detected using C_{L}'s comprising an antibody to the constant region of the antibody C_{A}.

### Pairing Components and Structures

In general, the members of a pairing component set utilized in the invention should meet certain criteria: 1) Each member of the component pair should have high specificity for its complement (i.e., low cross-reactivity) under the pairing conditions for the set; 2) Each member of the component pair should have a high affinity (binding constant) for its complement, or high avidity; 3) The pairing conditions for the individual pairing components should not preclude pairing for the other pairing components in the set; 4) In general, the chemistry and physical properties of all pairing components of the set should be predictable and well-understood, including the ability to control the charge of the individual pairing components of the set; 5) The pairing components of the set should generally be un-reactive with any IC's which may the used in the methods or compositions of the invention; and 6) The set should have a large number of possible pairing components which meet the above criteria. In general, nucleic acid oligomers and synthetic analogs with similar pairing and chemical properties are the best match for the above criteria. Thus, nucleic acid oligomers and synthetic analogs (such as amide-RNAs and p-RNAs) are preferred for use in as pairing components in the methods and structures of the invention.

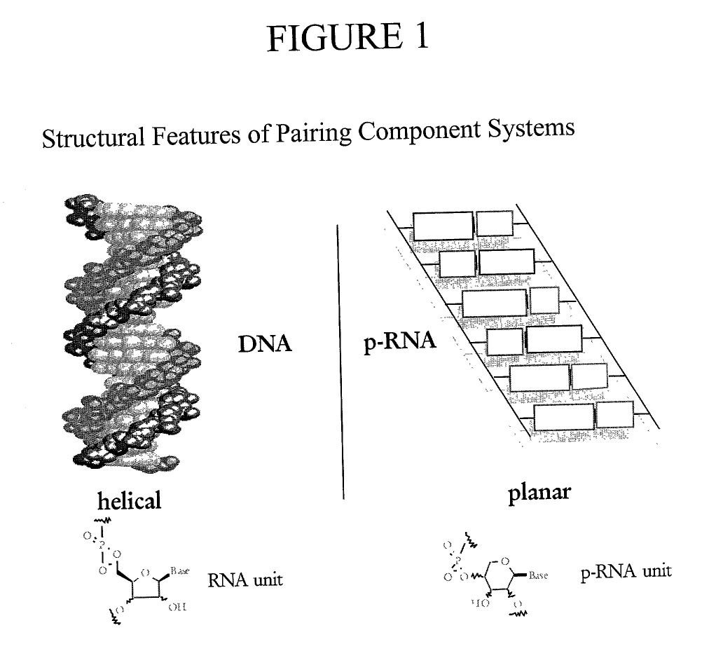

One type of a pairing component which can be used to form self-assembling intermolecular ligand binding structures is pyranosyl-RNA or p-RNA. p-RNA is a nucleic acid-like molecule in which the sugar group is a pentopyranose (see FIG. 1). (See Pitsch, S., et al., Helv. Chim. Acta, 76, pp. 2161-2183, 1993) The replacement of the normal deoxyribose (or ribose) with the pentopyranose sugar leads to a planar form for the hybridized double-stranded p-RNA (see FIG. 1). In many respects, such as Classical Watson-Crick type base pairing, enantioselective base pairing, and antiparallel strand orientation for double-stranded structure, the p-RNA molecule acts similar to nucleic acids.

However, some important p-RNA characteristics which distinguish the molecules from nucleic acids include: higher duplex stability and selectivity than DNA or RNA, the inability of p-RNA to base pair with DNA or RNA, and the fact that p-RNA duplexes form quasi-ladder planar structures, not the classical helix. The ability to create pairable molecules according to conventional Watson-Crick base-pairing rules, that also do not interact with native nucleic acids in solution, is very appealing when choosing the pairing components for use in the present invention. Thus, a preferred embodiment utilizes pyranosyl-RNA (“p-RNA”) as the self-assembling pairing component for attachment to the binding immunoreaction component.

The basic procedure for the synthesis and purification of pRNA is given in the Experimental Section (see Example 1). See also, WO 99/15539, entitled “Pentopyranosyl Nucleoside, and Production and Use of the Same”, filed Sep. 22, 1997; WO 99/15540, entitled “Method for Producing a Pentopyranosyl Nucleoside”, filed Sep. 22, 1997; WO 99/15541, entitled “Pentopyranosyl Nucleoside for Producing an Electronic Component, and Conjugates of Said Pentopyranosyl Nucleoside”, filed Sep. 22, 1997, incorporated herein by reference as if fully set forth herein. The p-RNA molecules can be derivatized with many of the same components and by many of the same procedures that have been developed for DNA and RNA modification. In particular, the phosphoramidite chemistries developed for nucleic acids are also very useful for both building p-RNA molecules and for derivatizing those molecules at a terminal phosphate. Thus, p-RNA can be derivatized (functionalized or modified) with biotin moieties, aromatic and aliphatic amine groups, aromatic and aliphatic thiol groups, aromatic and aliphatic aldehyde groups.

p-RNA can also be functionalized by incorporation of a tryptamine ribopyranosyl (I) phosphoramidite at the terminal position or anywhere within the sequence. See, e.g., WO 99/15542, entitled “Linker Nucleoside, and Production and Use of the Same”, filed Sep. 22, 1997, incorporated herein by reference as if fully set forth herein. The procedure for incorporation of a tryptamine ribopyranosyl (I) phosphoramidite into p-RNA is given in the Experimental Section. Thus, a p-RNA with can be further derivatized at a pendant tryptamine amino group, which is a suitable attachment site for the immunoreaction component. Alternately, the tryptamine may be derivatized with fluorophores, chromophores, biotin, chelates, amino acids, and peptides, proteins, streptavidin, nucleic acids (DNA/RNA), nanoparticles, and a variety of other molecules and structures.

p-RNA's functionalized with amines, thiols, aldehydes, and/or tryptamine (I) nucleotides can also be subsequently attached to solid supports and surfaces. Such surfaces include, but are not limited to, glass, silicon, plastics, nylon, nitrocellulose, ceramics, metals, metal oxides, polyacrylamide, other hydrogels, agarose, and other polysaccharides. p-RNA's can be functionalized at their 2′ or 4′ terminal positions or at any position within the sequence. Derivatization of p-RNA can be carried out via modification of the base moieties, sugars, or the phosphate groups, by utilizing synthesis schemes chemically analogous to those utilized with nucleic acids.

While p-RNA is preferred for use in the pairing components of the invention, another potentially useful group of pairing components includes cyclohexyl nucleic acids or CNA's (CNA-peptide pairing systems are disclosed in WO 99/15509, entitled “Cyclohexyl and Heterocyclyl Nucleoside Derivatives, Method for Producing These Derivatives, and the Use of the Derivatives and Their Oligomers or Conjugates in Pairing and/or Testing Systems, filed Sep. 22, 1997). CNA's have an uncharged backbone structure, which means that they could have advantages for forming pairing structures under low ionic strength conditions. However, it is still desirable that the entirety of the pairing component-immunoreactant complex carry a charge, in order to ensure electrophoretic mobility in the methods and compositions of the invention.

The more traditional deoxyribonucleic acids (DNA) and ribonucleic acids (RNA) are also useful as pairing components. These molecules have the advantages of familiarity and a large body of conventional chemistry techniques for modification and linkage to an IₙC_{B}. In addition, a wide variety of readily modified and pre-modified monomer units are commercially available, with fluorophores, succinimidyl ester linkers, biotin, and other labels already present on the nucleotide.

A primary drawback to the use of these molecules as pairing components is the possibility of interaction with native nucleic acids (genomic DNA, MRNA, other contaminating DNA or RNA) present in the sample, which may interfere with the assay results. In addition, if DNA assays for mutations, or the like, are to be run on the same sample, or on the same matrix, then care must be taken not to use sequences which may hybridize with either the probe or the nucleic acid analyte. Thus, any DNA or RNA sequence which is to be used as a pairing component should be checked against known sequences for the intended sample organism, and possible contaminating organisms, to prevent cross-hybridization. As the complete genomes several organisms are now available, such a comparison may often be accomplished by comparison with the organism's genome database. In addition, a comparison against all known sequences in one of the large international databases (e.g., SwissProt or GenBank) is recommended, to uncover possible splicing variants or contaminating organism sequences which may hybridize with the pairing component sequence.

In addition to the above noted nucleic acid analogs and nucleic acids, other synthetic analogs may be utilized as pairing components in the invention. Methylphosphonate nucleic acid analogues, phosphorothioate nucleic acid analogues, phosphorodithioate nucleic acid analogues, peptide nucleic acids (PNA), amide nucleic acid analogs and other synthetic nucleic acids may be used. In these structures, the sugar-phosphate backbone is modified or wholly replaced with another backbone structure, while the usual nucleotide bases are typically retained. Thus, the nucleic acid base moieties serve to form the intermolecular pairing system in agreement with classical hydrogen-bonding based hybridization, according to normal Watson-Crick rules. Synthetic analogs that do not interact with DNA and RNA are preferred over those that do, for the reasons noted above.

### Binding Immunoreaction Component—Pairing Component Structures Utilized in the Invention

In the methods and compositions of the invention, one member of the component pair (P₁) is derivatized for attachment to the active matrix test site, and the other member of the pair (P₁′) is derivatized for attachment to the binding immunoreaction component (I₁C_{B}). In particular, streptavidin, avidin, and biotin are useful non-covalent derivitizations for use to attach one of the pairing component members to the test site, although other covalent linkage chemistries or ligand moiety linkages may be used. Any suitable linkage chemistry may be used to attach the other member of the component pair: both streptavidin/biotin linkage to an antibody and iodoacetyl linkage to a free sulfhydryl on an antibody fragment have proven effective.

The pairing component member complement, Pₓ′ (e.g., a p-RNA sequence) is functionalized to either covalently or noncovalently attach to the surface of a test site on the microelectronic array. By way of example, the p-RNA pairing component complement members can be functionalized with a biotin moiety and then attached to the microelectronic array test sites via streptavidin incorporated in the permeation layer of test sites. It is also possible to covalently attach or immobilize p-RNA sequences that have been functionalized with amines, thiols, aldehydes, carboxyl groups, hydrazines, azido groups, and with phenylboronic acid. Generally, the pairing member p-RNA sequence is functionalized by a phospho linkage at either its 4′ or 2′ terminal position when attachment to a solid support is the objective. However, it is possible to functionalize the p-RNA at any position in its sequence for attachment to solid supports, as long as its ability to pair with the other member of the component pair is not inhibited.

The other member of each pairing component, such as a p-RNA sequence, is generally covalently bound to each IₙC_{B} through an appropriate organic linkage chemistry. For example, the use of an iodoacetyl group on a derivatized pRNA to link the PRNA to the I₁C_{B} through the sulfhydryl of a cysteine residue on a proteinaceous IₙC_{B} (antibody, antibody fragment, or antigen.) Covalent coupling of the peptide can be achieved by either using a functional group provided by one or more of the amino acids in the peptide itself, or by incorporating additional functionalization into the peptide sequence. Functional groups which may be provided by one or more of the amino acids in the peptide sequence itself include Cysteine (thiol), Lysine (amino), Serine (hydroxyl), Tyrosine (hydroxyl), Glutamate (carboxyl), Aspartate (carboxyl), the N-terminus (amino), and the C-terminus (carboxyl).

Functional groups for coupling reactions that can be incorporated into p-RNA include: tryptamine nucleotides, amines, thiols, aldehydes, hydroxyl (ribose), carboxyl, phosphate, maleimides, haloalkyls (iodoalkyl, chloroalky, and bromoalkyl) and a number of others. One particular method relevant to this invention for coupling a peptide via the cysteine thiol group involves using an antibody or antibody fragment with a reduced free cysteine which is then reacted with an iodoacetyl group on the pRNA terminus. Cysteine containing IₙC_{B}'s may also be linked to the pairing component member via the primary amine of a tryptamnine residue. Additionally, in some cases it may be desirable to have IₙC_{B}-Pₓ structures in which the immunoreactant structures are separated from the p-RNA structure. This would be important when it is necessary to bind larger analyte immunoreaction component molecules and structures (cell surfaces, etc.) Thus, the use of so-called spacer groups is also incorporated into this invention. Such spacer groups include, but are not limited to, a short run of amino acids (—glyₙ—), aliphatic chains (—CH₂—CH₂—CH₂—CH₂—), polyalkylene glycols, and polysaccharide structures. Spacer groups may be added chemically to the pairing component member, or may be engineered into a recombinant IₙC_{B} protein structure. Similarly, residues for attachment, such as cysteine, may be engineered into the IC protein structure. Several such modifications for the constant regions of engineered recombinant antibodies are known in the protein design arts. Spacer groups can be designed to provide either rigid or flexible intervening structures between the pairing component member and the I₁C_{B}.

However, non-covalent attachments are also useful, and biotin/avidin (or biotin/streptavidin) interactions may be more convenient for a particular assay format. For instance, in embodiments where the pairing components are provided in a kit for use with antibodies supplied by the investigator, each pairing component member to be attached to the IₙC_{B} may be derivatized with a biotin moiety (e.g., p-RNA-biotin). Then, the investigator may simply utilize standard streptavidin-functionalization chemistries to add a streptavidin moiety to an antibody or antigen, and then couple the resulting conjugate with the provided Pₓ-biotin. The resultant Pₓ-biotin-streptavidin-IₙC_{B} may then be used in the immunoassay methods of the invention. An example of such an assay is given in Example 3. However, when utilizing these formats, it is preferred that another type of attachment be utilized to bind the first Pₓ′ complement to the test site on the active electronic matrix device.

### Electronic Addressing of Arrays, and the Formation of p-RNA Complement Pairing Component Arrays

In the active electronic array embodiments of the invention, electronic addressing is utilized to selectively concentrate reactants at the individually activated test sites. In short, the electrode under the test site is appropriately biased (positive when addressing nucleic acid or pRNA pairing component members), creating an electric field which draws the reactants to the test sites. Thus, the basic principle behind electric addressing is the use of free-filed electro-kinetic motion to redistribute charged species to the area around the activated, or biased, test site.

Briefly, the charged species are present in an approximately uniform dispersion in the solution which is applied to the active electronic matrix array. For the purposes of illustration, one may take a p-RNA oligomer member of a pairing component, which has been derivatized with a biotin. The p-RNA oligomer is thus present at a relatively low concentration over the volume of the solution, and over the surface of the permeation layer, which in this illustration contains streptavidin. When a positive bias is applied to a row of test sites (with an appropriate negative bias applied at another reference electrode), an electric field is created which draws all negatively charged molecules in solution towards the electrodes under the positively biased row of test sites. The p-RNA oligomer is then transported along the electric field lines and then concentrated at the test sites. Thus, the concentration of the derivatized oligomer is greatly increased at the test sites, which the concentration of the oligomer is decreased in the rest of the solution. This leads to an increased frequency of binding events at the test site, greatly accelerating the binding of the oligomer at the test site.

Thus, the active electronic microarrays for use in the methods of the invention may be readily electronically addressed with p-RNAs at particular test sites. Generally, the addressing solution contains between 1 to 100 nM of the biotinylated p-RNA sequence in a 25-100 mM histidine (zwitterionic) solution. Because of the electric field strength is affected by the conductance of the connecting liquid, low conductance zwitterionic buffers (such as 50-250 mM γ-amino butyric acid, histidine, methylhistidine, carnosine, glycine, β-alanine, taurine, cysteine, lysine or other amino acids) are preferred for use in the electronic addressing methods of the invention. Alternatively, other low conductance “good” buffers such as HEPES, pyridine, imidazole, or collidine buffers may be used. Preferred low conductance buffer formulations are described at length in U.S. Pat. No. 6,051,380, entitled “Methods and Procedures for Molecular Biological Analysis and Diagnostics,” incorporated above. Electronic addressing is carried out at a current of about 200 nA to 6 nA (to the positively biased site) for a period of 60 to 120 seconds. In general, these planar microchip devices are operated in a low range of currents (˜10 nA to ˜5 μA) and voltages (˜1.2 to 5.0 volts). Alternatively, mechanical means of selectively attaching pairing component members may be used. These include ink jetting, micro-pipetting, microcapillary deposition and other techniques, many of which are know to those in the nucleic acid microarray art. In general, mechanical methods are less preferred than electronic methods because electronic methods are more easily utilized after the assembly of the electronic matrix device. However, mechanical means may be appropriate, for instance, in cases where a large number of active arrays with a standardized set of pairing component members is produced.

Surprisingly, applicants have found that the electronic addressing technique can be used not only for nucleic acid-like molecules, such as p-RNAs, which have a relatively high charge-to-molecular weight ratio, but also for molecules that are much larger, and less highly charged. Particularly, applicants have found that by adding a small nucleic acid sequence to large macromolecules, e.g., a p-RNA pairing component, such molecules may also be efficiently and specifically addressed to the test sites of the active electronic matrix array devices used in the present invention. Thus, an P₁-I₁C_{B} may be readily addressed electronically to a particular biased test site, or set of test sites, in a matter of minutes. This allows for dramatic economic advantages, as the amount of reactants in solution necessary to address a binding immunoreaction component to a -particular test site is far less than that required to bind a similar amount to the test site in a similar time under passive conditions.

In addition to P₁-I₁C_{B} reagents, applicants have found that entire immuno complexes, complete with analyte and labeling components, may be similarly electronically addressed. Thus, and entire P₁-I₁C_{B}-I₁C_{A}-I₁C_{L1}-I₁C_{L2} complex may be rapidly electronically addressed to a test site from a complex sample immunoreaction mixture. Surprisingly, the interactions between the immunoreaction components are relatively unaffected by the low-conductance buffer conditions preferred for use with electronic addressing techniques.

Finally, applicants have also found that immunoassays may be readily developed with conditions which allow the selective electronic addressing to biased test sites of analytes, or analytes-labeling component complexes, from the sample immunoreaction mixture. This allows for the selective activation of different sets of test sites containing the same IₙC_{B} reagents, without significant cross-talk between samples addressed to different sets of sites. Obviously, this technique is somewhat limited by the charge characteristics of the analyte immunoreaction components to be addressed. However, reaction conditions, especially pH, may be readily modified to convey a slight charge on most antigen or antibody analytes; the Examples clearly demonstrate the utility of such assays for sets of two, three, four, and even larger groups of analytes in a sample. Furthermore, this technique is be more widely applicable when labeling immunoreaction components with defined charge characteristics are used. For instance, recombinant antibodies for use in IₙC_{L}'s may be designed that contain a number of additional or substitute glutamic acid residues in their constant regions, conveying a significant negative charge to each IₙC_{A}-IₙC_{L} complex. This would allow for more ready addressing of analyte components of different charges under a commonly suitable immunoreaction condition.

### Basic Immunoreaction Assay Methods of the Invention and their Use in Proteomic Analysis

As discussed in the Summary of Invention, multiple immunodiagnostic assays can be developed by preparing specific pairing component-binding immunoreaction component conjugates, as described above, which are then selectively electronically addressed to a microelectronic or other array type device or substrate. The two basic formats of these methods of the invention are 1) off-chip formation of the immunoreaction complex containing the pairing component, with subsequent addressing of the complex to the proper test sites on the active electronic matrix, and 2) pre-immunoreaction electronic addressing of the binding immunoreaction component to specific test sites on the active electronic matrix, based on the pairing component attached to the binding immunoreaction component, with subsequent passive or electronic incubation of the sample analytes with the arrayed binding immunoreaction components. For the facility of the discussion of these basic formats, and their multiplexing, a typical sandwich assay in which the pairing components are p-RNA, the C_{B}'s are antibodies, and the C_{A}'s are antigens, will be used, although these may be any of the molecular species discussed above. In addition, as both basic assay methods are suitable for use with either direct detection (sandwich) or competitive assay formats, the discussion of these formats is reserved for the Formats section, infra, and this preliminary discussion is limited to the formation of the basic immunoreaction complex, IₙC_{A}-IₙC_{B}-Pₓ-Pₓ′, for detection at the test site.

In the first format, specific complementary pairing component p-RNA sequences (capture sequences) are pre-addressed to the array, as described above. In first step of the assay, a set of IₙC_{B}-Pₓ conjugates are reacted in solution with samples containing the target analytes (IₙC_{A}), where a different IₙC_{B}-Pₓ is provided for each analyte. Any material which may contain the analyte of interest may be utilized as a “sample.” Exemplary animal derived materials for testing as the sample include bodily fluids such as whole blood, serum, plasma, saliva, lymph, ascites, and urine, as well as stool, tissue samples or tissue homogenates, and extracts or dilutions of any of these. Other biological samples which may be used include cell cultures or culture supernatants, saps, secretions, mucus. Food or water samples may be of interest for contaminant testing.

The IₙC_{A}-IₙC_{B}-Pₓ complexes are then selectively immobilized by electronic hybridization to their complementary pairing component members on the array. In this manner, the immunoreaction complexes are each addressed to a test site in the active matrix, “resolving” the products of the immunoreaction, and allowing the detection of the presence of the immunoreaction complex at the test site. In this type of multiplexed embodiment, each Pₓ should be different, in order to allow the mixture of immunoreaction complex species in the sample immunoreaction mixture to be resolved. The immunoreaction mixture is then contacted with the active electronic matrix, which has been prepared with a plurality of test sites containing appropriate pairing member complements (Pₓ′). The test sites containing the complements are electrically biased to promote the pairing of the members of the pairing components and thus creating addressed immunoreaction complexes, IₙC_{A}-IₙC_{B}-Pₓ-Pₓ′, attached to their respective test sites. Detection may then be achieved by the incorporation of a labeled immunoreaction (I₁C_{L}) component, or by the detection in the decrease in the incorporation of a labeled analyte standard (IₙC_{A}*).

Although generally illustrated above for the case where the investigator wishes to simultaneously form multiple immunoreaction complexes with multiple analyte immunoreaction components in the sample immunoreaction mixture, and then resolve the immunoreaction complexes onto the test sites of the active electronic matrix, the method may also be used with a single analyte. However, multiplex assay methods are preferred, and this takes full advantage of the resolving power of the methods of the invention. In these embodiments, three, four, five, and even ten or more, analyte immunoreaction components may be resolved and detected.

In addition, these methods may be multiplexed by the selective addressing of different sample immunoreaction mixtures to different sets of test sites. In these embodiments, the active electronic matrix array is prepared with two or more sets of test sites containing a set of complementary pairing components (P₁-Pₓ′). For illustration, it is easier to consider a limited multiplex reaction with two analytes. In this embodiment, the two immunoreaction complexes I₁C_{A}-I₁C_{B}-P₁ and I2C_{A}-I2C_{B}-P₂ from a first sample immunoreaction may be selectively electronically addressed to test sites 1A and 1B in a first row of the active electronic matrix, the first set of P₁′ and P₂′ sites. Then, a second immunoreaction mixture may be resolved by biasing test sites 2A and 2B , the second set of P₁′ and P₂′ sites, in a second row, thus attaching the second sample's immunoreaction complexes to 2A and 2B . The presence of the analyte immunoreaction components in each sample may then be detected by, for instance, incubating the entire active electronic matrix surface with labeled immunoreaction components. Because the individually controlled test sites may be made highly selective for the hybridization or binding of the pairing components, three, four, five, ten, or several dozen samples may be analyzed in this manner, and compared side by side on the active matrix.

In the second basic embodiment of the immunoassay methods of the invention, the binding immunoreaction components IₙC_{B}-Pₓ are first addressed to test sites on the active electronic matrix, and then allowed to reacted with the analytes by incubation with the sample. An active electronic matrix array is first prepared with one or more sets of test sites containing complementary pairing components (P₁′-Pₓ′). Then, a set of binding immunoreaction component-first pairing component member complexes (I₁C_{B}-P₁-IₙC_{B}-Pₓ) is then electronically addressed to the test sites. The pairing component members then selectively pair, creating an addressed binding immunoreaction component complexes, I₁C_{B}-P₁-P₁′ through IₙC_{B}-Pₓ-Pₓ′, attached to the test sites. This is a concurrent electronic addressing example, in which all of the binding immunoreaction components are simultaneously addressed to their respective test sites.

These methods may also be utilized to electronically address at least two sets of immunoreaction components to at least two sets of test sites with the same set of pairing components by sequential electronic addressing of sets of IₙC_{B}-P₁-IₙC_{B}-Pₓ to sets of test sites containing P₁′-Pₓ′. To illustrate these methods, a first set of immunoreaction component-pairing component complexes, I₁C_{B}-P₁ and I2C_{B}-P₂, may be selectively addressed to test sites 1A and 1B containing P₁′ and P₂′, in a first row of the active electronic matrix. Then, a second set of immunoreaction component-pairing component complexes, I₃C_{B}-P₁ and I₄C_{B}-P₂, may be addressed by biasing test sites 2A and 2B , containing P₁′ and P₂′, in a second row on the same active electronic matrix. Thus, by utilizing the selective activation of sets of test sites in two rounds, a set of two pairing components may be utilized to address four binding immunoreaction components to four test sites. Analogously, a set of ten pairing components may be utilized to address 100 different immunoreaction components to 100 test sites in ten rounds of electronic addressing. As is evident from these methods, it is not necessary for each binding immunoreaction component for the detection of each analyte to have its own pairing component. By simply utilizing several sequential electronic biasing steps, several sets of sets of test sites containing P₁′-Pₓ′, allowing for the rapid creation of large antibody or antigen arrays for screening against a sample.

The active electronic matrix is then incubated, either electronically or passively, with a sample which may contain an analyte immunoreaction component I₁C_{A}, thus forming an attached immunoreaction complex I₁C_{A}-I₁C_{B}P₁-P₁′. The attached complex may then be detected at the test site. In addition to accelerated and selective pairing component addressing of binding immunoreaction component-pairing component complexes, the analyte immunological components may also be electronically addressed to the test sites in the methods of the invention, depending on the charge characteristics of the analyte. In these embodiments of the methods of the invention, the binding immunoreaction component-pairing component complexes are first electronically addressed to a set of test sites in the active electronic matrix, as described above. Then, the sample is electronically incubated with the matrix by electronically biasing the test sites so as to concentrate any analyte immunoreaction components present in the sample at the activated test sites. The analytes in the sample rapidly react with the binding immunoreaction components, forming immobilized immunoreaction complexes. These may then be detected by any of the means described above.

In addition to accelerated reaction, the electronic incubation methods used in the immunoassays of the invention also allow the selective addressing of sample analyte immunoreaction components to the test sites of the active electronic matrix. To illustrate these methods, a set of immunoreaction component-pairing component complexes, I₁C_{B}-P₁ and I₂C_{B}-P₂, may be selectively addressed to test sites 1A and 1B, containing P₁′ and P₂′, in a first row of the active electronic matrix, and to test sites 2A and 2B , also containing P₁′ and P₂′, in a second row of the active electronic matrix. A first sample is then contacted with the matrix while test sites 1A and 1B are appropriately electronically biased, selectively concentrating and reacting the analytes I₁C_{A} and I₂C_{A} at those test sites. A second sample may then be contacted with the matrix while test sites 2A and 2B are appropriately biased, selectively concentrating and reacting the analytes I₁C_{A}and I₂C_{A} from the second sample at those test sites. In this fashion, several samples may be analyzed utilizing selected portions of the same active electronic matrix with negligible or insignificant side reactions in the unselected portions of the matrix, even though the unselected portions contain the same attached binding immunoreaction components.

These two basic formats are illustrated in the following Examples. Example 3 describes a procedure where complementary p-RNA constructs were used as pairing component members for a protein conjugate consisting of streptavidin linked to a goat anti-human IgG F(ab′)₂ antibody. p-RNA No. 81 was used to provide a capture sequence for p-RNA No. 80 by binding the biotin of p-RNA No. 81 to streptavidin-agarose in the permeation layer of a test site in an active electronic matrix array. The biotin of p-RNA No. 80 was then used to bind to a streptavidin-goat anti-human IgG F(ab′)₂ antibody conjugate (made by conventional immunochemistry means, and available commercially). The goat anti-human F(ab′)₂ antibody-p-RNA complex was used to capture its specific antigen target, a human IgG. This represent just one of a number of methods by which antibodies can be modified with specific p-RNA molecules and addressed to a microelectronic or other array device. Likewise, in Example 3, complementary p-RNAs #54 and #79 were used to form another pairing component to illustrate a basic form of the second method embodiment. In this case p-RNA #54 was immobilized to the streptavidin-agarose permeation layer overlaying the test site. p-RNA #79 was hybridized to its complementary strand #54 and the 4′ biotin of #79 was used to immobilize the streptavidin-goat anti-human IgG conjugate. The goat anti-human F(ab′)2 antibody was then used as an immunosorbent to capture its target, human IgG.

The selectivity of the respective p-RNA sequences as pairing components (either #81 or #54) was evidenced that when the complementary strands of p-RNA (either #80 or #79, respectively) were bound to the streptavidin-goat anti-human antibody protein conjugate in solution, and then hybridized to an APEX chip containing the captures, the p-RNAs attached to the protein conjugate bound only to their respective immobilized complementary pairing component strands. This demonstrates the basis for enabling geographical sorting, or electronic addressing resolution, of immunological reagents which are simultaneously present in a complex immunoreaction solution.

Example 8 demonstrates these methods, achieving a simultaneous multiplex immunoassay combined with resolution of the immunoreaction complexes for discrete analyte detection. Using the complementary pairs of p-RNA sequences as pairing components, and a second protein conjugate consisting of streptavidin chemically coupled to a murine monoclonal antibody against the α-subunit of human Chorionic Gonadotropin, a simultaneous immunological detection of two different antigens was accomplished. The p-RNA pairing component members successfully differentiated between their respective complementary strands such that the two antigen targets were differentially detected by the complementary p-RNA pairing component members on their respective test sites.

This demonstrates the capability for multiple simultaneous immunological reactions to be performed in solution coupled with individual detection of the specific antigen targets of each of the individual immunological reactions. The separation of antigen target detection is accomplished by employing the selectivity of p-RNA strands for their respective complements to achieve selective antigen target detection.

Example 5 demonstrates a similar multiplex analysis utilizing the second basic format of the methods of the invention. In this example, p-RNA sequences 1a and 1b, and 81 and 80, were utilized to as pairing components to specifically electronically address anti-myoglobin and anti-CKMB antibodies to specific test sites. Upon contacting samples containing the antigens, and electronic biasing of the test sites, specific electronic addressing of the antigens to the biased test sites was achieved. This demonstrates the feasibility of using electronic incubation to selectively address antigens from several samples to biased sets of test sites on the same active electronic matrix array, with little, if any, cross-talk between samples.

### Electronic Stringency in the Methods of the Invention

In addition to the advantages of electro-kinetic movement and concentration of reactants, microelectronic arrays also provide the added parameter of selective electric field stringency control at each test site on the array. Thus, microelectronic arrays have the potential to achieve higher order specificity for the immunoassay reactions, and the hybridization-interactions of pairing member components, by varying the electronic environment at the individual test sites. Thus, in addition to the classical stringency parameters which include: temperature, pH, ionic strength, and chemical agents (detergents, denaturants, chaotropic agents), the application of an electric field stringency to immunoreaction complex formation provides a novel and powerful parameter for the selective addressing of binding immunoreaction components, and even entire immunoreaction complexes or analyte immunoreaction components from sample mixtures.

Active microelectronic chip/array technologies have been demonstrated in the above referenced patents which provide the capability for rapid multiplex hybridization and electronic stringency for improving hybrid selectivity. Briefly, the hybridization of nucleic acids, and their synthetic analogs, is often done under conditions where some mismatching between nucleotide bases occurs. When array formats are used, temperature, pH, and the ionic strength of the solution are often chosen to ensure that the least stable hybridization pair will form, allowing some of the more stable mismatched pairs to remain hybridized in the array. By reversing the electronic bias of the test site, these mismatched hybrids may be destabilized and removed from the array. Similarly, reversed bias may be utilized to destabilize immunoreaction complexes containing charged immunoreaction components. Electronic stringency may be carried out using a current or voltage that is less than, equal to, or even greater than that used for the electronic addressing of the pairing components or immunoreaction components.

In addition to electronic stringency, wherein materials partially bound to a pairing component or immunoreaction component are removed, a technique called “electronic washing” is also useful in the methods of the invention. In this technique, reagents that are non-specifically bound at the test site (e.g., partially embedded in the permeation layer hydrogel matrix) are removed by a short reversed bias electric field at the test site. This will often reduce or eliminate background signal from non-specifically bound labeled molecules. Electronic washing is usually done at a lower voltage or current than used for any electronic addressing steps, and over a shorter period of time.

### Assay Format Options for Use with the Basic Immunoassay Methods of the Invention

For the detection and measurement of analytes large enough to have epitopes for 2 or more different antibodies (e.g., most proteins, carbohydrates, and other biological molecules), it is often desirable to construct direct sandwich-type assays on the chip. As discussed in the basic methods section above, the active electronic matrix array is first prepared for use in the assay by attaching a complementary binding component member Pₓ′, e.g., p-RNA oligomers, to each test site to be used in the array. This may be easily accomplished by electronic addressing of the derivatized p-RNA oligomers (e.g., biotin-p-RNA's) to the test sites for binding with attachment moieties in the permeation layer (e.g., streptavidin-agarose). Alternatively, the complementary pairing component members could be directly spotted onto the active matrix array. This approach is less preferred, but may be useful for mass-produced arrays with rows of standard complementary pairing component members.

The Pₓ-IₙC_{B} (pRNA-Ab conjugates), IₙC_{A} (antigen analytes in the sample), and an IₙC_{L} (Ab2*, or labeled 2^{nd} antibody) are then added sequentially or simultaneously depending on the particular requirements of the assay. C_{B}, C_{A}, and C_{L} may be any of the above described immunoreaction component molecules, including be antibodies, DNA or RNA aptamers, metal chelators, or protein binding partners. Although an antigen is the middle of the “sandwich” for the purposes of illustration below, these assays may also be formatted wherein C_{B} is and antigen, C_{A} is an antibody, and C_{L} comprises another antibody to the analyte antibody. The Pₓ-IₙC_{B} (pRNA-Ab conjugates), IₙC_{A} (antigen analytes in the sample), and an IₙC_{L} (Ab2*, or labeled 2^{nd} antibody) may be added as individual components or as simple or complex mixtures. Washes may be added between the steps shown, as necessary.

In the table below, left arrows indicate that the respective species is introduced to the chip containing the previously constructed binding element. Modes D1-D8 show the basic options for a single antigen assay with sequential addition of 1) antigen analyte in the sample and then 2) a labeled second antibody. Modes D9-D12 show the basic options for the pre-application incubation of the antigen analyte with the binding first antibody. Modes D13-D16 show the basic options for the pre-application incubation of the antigen analyte with the second detection antibody. Modes D17 and D18 show the basic options for the pre-application incubation of the antigen analyte with both the first binding antibody and the second detection antibody.

TABLE I
Direct Assay Formats

| Mode | Pre-Step | 1^{st} Step | 2^{nd} Step | 3^{rd} Step |
|---|---|---|---|---|
| | Complement | | | |
| | p-RNA (P₁′) | p-RNA-Ab1 (P₁-I₁C_{B}) | Ag (I₁C_{A}) | Ab2* (I₁C_{L}) |
| D1 | electronic | ←passive | ←passive | ←passive |
| D2 | electronic | ←passive | ←electronic | ←passive |
| D3 | electronic | ←electronic | ←passive | ←passive |
| D4 | electronic | ←electronic | ←electronic | ←passive |
| D5 | electronic | ←passive | ←passive | ←electronic |
| D6 | electronic | ←passive | ←electronic | ←electronic |
| D7 | electronic | ←electronic | ←passive | ←electronic |
| D8 | electronic | ←electronic | ←electronic | ←electronic |
| | Complement | [p-RNA-Ab1-Ag] | | |
| | p-RNA (P₁′) | P₁-I₁C_{B}-I₁C_{A} | Ab2* (I₁C_{L}) | |
| D9 | electronic | ←passive | ←passive | |
| D10 | electronic | ←passive | ←electronic | |
| D11 | electronic | ←electronic | ←passive | |
| D12 | electronic | ←electronic | ←electronic | |
| | Complement | p-RNA-Ab1 | [Ag-Ab2*] | |
| | p-RNA (P₁′) | P₁-Ihd 1C_{B} | I₁C_{A}-I₁C_{L} | |
| D13 | electronic | ←passive | ←passive | |
| D14 | electronic | ←passive | ←electronic | |
| D15 | electronic | ←electronic | ←passive | |
| D16 | electronic | ←electronic | ←electronic | |

| | Complement | [pRNA-Ab1-Ag-Ab2*] |
|---|---|---|
| | p-RNA (P₁′) | P₁-I₁C_{B}-I₁C_{A}-I₁C_{L} |
| D17 | electronic | ←passive |
| D18 | electronic | ←electronic |

In addition to direct assay formats, competitive assays may be constructed in various ways to modulate the sensitivity and/or dynamic range of the assay, or to minimize the influence of potential cross-reacting antigens. As noted above, the first step in the assays is assembling an array of p-RNA Pₓ′ containing test sites, preferably by electronic addressing. The Pₓ-IₙC_{B} (pRNA-Ab conjugates), IₙC_{A} (antigen analytes in the sample), and IₙC_{A}* (a known concentration of labeled antigen, which competes for binding sites with the antigen analyte in the sample) are then added sequentially or simultaneously depending on the particular requirements of the assay. The complementary Pₓ-IₙC_{B}, IₙC_{A}, IₙC_{A}* may be added as individual components or as simple or complex mixtures (i.e., electronically addressing all Pₓ-IₙC_{B} simultaneously, rather than sequentially. Washes may be added between the steps shown as necessary.

In the table, Modes C1-C4 show assay formats in which labeled and unlabeled (sample/analyte) antigens are added to the array simultaneously. Modes C5-C8 show the pre-application incubation of the binding antibody with the unlabeled analyte. Modes C9-C12 show the pre-application incubation of the binding antibody with the labeled antigen standard. Modes C13-C20 show the delayed addition of the unlabeled antigen analyte. Modes C21-28 show the delayed addition of the labeled antigen standard. Note that in modes C9-C20, the incubation of the labeled antigen standard with the binding antibody is usually not allowed to come to equilibrium before the subsequent addition of the unlabeled analyte antigen, or a subsequent prolonged incubation period is used to equilibrate or partially equilibrate with the unbound analyte antigen in solution.

TABLE II
Competitive Assa Formats

| Mode | Pre-Step | 1^{st} Step | 2^{nd} Step | 3^{rd} Step |
|---|---|---|---|---|
| | Complement | p-RNA′-Ab | [Ag + Ag*] | |
| | p-RNA (P₁′) | P₁-I₁C_{B} | I₁C_{A} + I₁C_{A}* | |
| C1 | electronic | ←passive | ←passive | |
| C2 | electronic | ←passive | ←electronic | |
| C3 | electronic | ←electronic | ←passive | |
| C4 | electronic | ←electronic | ←electronic | |
| | Complement | [p-RNA-Ab-Ag] | Ag* | |
| | p-RNA (P₁′) | P₁-I₁C_{B}-I₁C_{A} | I₁C_{A}* | |
| C5 | electronic | ←passive | ←passive | |
| C6 | electronic | ←passive | ←electronic | |
| C7 | electronic | ←electronic | ←passive | |
| C8 | electronic | ←electronic | ←electronic | |
| | Complement | [p-RNA-Ab-Ag*] | Ag | |
| | p-RNA (P₁′) | P₁-I₁C_{B}-I₁C_{A}* | I₁C_{A} | |
| C9 | electronic | ←passive | ←passive | |
| C10 | electronic | ←passive | ←electronic | |
| C11 | electronic | ←electronic | ←passive | |
| C12 | electronic | ←electronic | ←electronic | |
| | Complement | p-RNA-Ab | Ag* | Ag |
| | p-RNA (P₁′) | P₁-I₁C_{B} | I₁C_{A}* | I₁C_{A} |
| C13 | electronic | ←passive | ←passive | ←passive |
| C14 | electronic | ←passive | ←electronic | ←passive |
| C15 | electronic | ←electronic | ←passive | ←passive |
| C16 | electronic | ←electronic | ←electronic | ←passive |
| C17 | electronic | ←passive | ←passive | ←electronic |
| C18 | electronic | ←passive | ←electronic | ←electronic |
| C19 | electronic | ←electronic | ←passive | ←electronic |
| C20 | electronic | ←electronic | ←electronic | ←electronic |
| | Complement | pRNA′-Ab | Ag | Ag* |
| | p-RNA (P₁′) | P₁-I₁C_{B} | I₁C_{A} | I₁C_{A}* |
| C21 | electronic | ←passive | ←passive | ←passive |
| C22 | electronic | ←passive | ←electronic | ←passive |
| C23 | electronic | ←electronic | ←passive | ←passive |
| C24 | electronic | ←electronic | ←electronic | ←passive |
| C25 | electronic | ←passive | ←passive | ←electronic |
| C26 | electronic | ←passive | ←electronic | ←electronic |
| C27 | electronic | ←electronic | ←passive | ←electronic |
| C28 | electronic | ←electronic | ←electronic | ←electronic |

For proteomic applications, it may also be useful to construct active matrix arrays for detecting the presence of known or unknown proteins in a sample that have certain physio-chemical properties. These types of arrays may be constructed to be less specific than antibody or antigen epitope arrays, allowing for selection of proteins based on their properties, rather than identity. By using a non-proteinaceous binding element such as an aptamer, wide range of proteins may be detected simultaneously using simple or sophisticated general protein detection methods, which may be chemical or physical (for example protein stains, surface plasmon resonance, front surface reflectance IR spectroscopy, or MALDI mass spectrometry).

In these methods, the first step in the assays is assembling an array of p-RNA Pₓ′ containing test sites, preferably by electronic addressing. The Pₓ-IₙC_{B} (PRNA-aptamer conjugates), IₙC_{A} (protein analytes in the sample), and I_{U}C_{L} (a universal protein binding agent or dye, which binds to all or most captured proteins) are then added sequentially or simultaneously depending on the particular requirements of the assay.

TABLE III
Protein Interaction Detection Formats

| | | 1^{st} Step | | 3^{rd} Step |
|---|---|---|---|---|
| | Pre-Step | p-RNA- | 2^{nd} Step | Protein Detection |
| | Complement | Aptamer | Sample | Reagent |
| Mode | p-RNA (P₁′) | Pₓ-IₙC_{B} | IₙC_{A} | I_{U}C_{L} |
| P1 | electronic | ←passive | ←passive | ←passive |
| P2 | electronic | ←passive | ←electronic | ←passive |
| P3 | electronic | ←electronic | ←passive | ←passive |
| P4 | electronic | ←electronic | ←electronic | ←passive |
| P5 | electronic | ←passive | ←passive | ←electronic |
| P6 | electronic | ←passive | ←electronic | ←electronic |
| P7 | electronic | ←electronic | ←passive | ←electronic |
| P8 | electronic | ←electronic | ←electronic | ←electronic |

As is evident from this discussion, the assay methods of the present invention are widely adaptable to most immunoassay formats, and also to similarly formatted solid-phase binding assays. Thus, the above examples are intended merely to illustrate possible formats for using the versatile methods of the invention. Alternate formats may be readily devised by one of ordinary skill in the immunoassay and biochemical arts, and are also considered to be within the scope of the present invention.

### Multiplexed Genomic, Gene Expression and Proteomic Analysis on a Single Active Electronic Matrix Array for the Diagnosis and Classification Disease

The power and flexibility of the use of pairing components to rapidly resolve multiplex immunoreactions by electronic addressing of the pairing component containing immunoreaction complexes is by now apparent. However, the active electronic matrix devices, buffers, and addressing procedures used in the proteomic analysis methods of the invention were originally designed for nucleic acid analyses and manipulation. Thus, the contemporaneous use of the same active electronic matrix device for proteomic and genomic and/or gene expression analysis is made feasible by the methods of the invention. In general, such multi-genre analysis (meaning the simultaneous analysis of multiple families of molecules, i.e. nucleic acids and proteins), has been thought to be too difficult, or even impossible, because the conditions necessary for efficient nucleic acid binding on passive arrays (e.g., high salt) and those necessary for the efficient binding of antibodies to their antigens are too disparate. Because the active electronic matrix methods of the invention do not require high salt conditions for the efficient binding of nucleic acids, and because the active electronic addressing methods are able to efficiently resolve immunoreactions after the antigen-antibody binding event has occurred (i.e., when the bound complex is in a very stable form), both nucleic acids and proteins may be easily assayed on the same active electronic matrix array device.

Furthermore, as both the proteomic and nucleic acid methods are facilely multiplexed, multiple proteomic and genomic/gene expression targets may be assayed simultaneously on the same electronic matrix array. In addition, the individually controllable nature of the test sites of the active matrix array allows for the multiplexing of multiple samples from multiple sources on the chip. Thus, on a 100-site chip, the level of 10 proteins and the level of expression of 10 genes from 5 sources' samples' may be ascertained, and compared side-by-side, on the same platform, using the same fluidic and electronic controls for the device. Although these analyses will require the processing of at least two samples from a source (one for protein analysis, one for nucleic acids), multi-sample analysis is often desirable for the two types of analysis. For example, proteomic analysis is limited by the fact that proteins are not amenable to enzymatic amplification; thus, a large sample is often necessary to collect a detectable amount of relatively rare proteins. Thus, secreted protein markers in blood, serum, plasma, urine, lymph, ascites, or other bodily fluids are often studied. Conversely, these fluids are practically useless for genetic or gene expression analysis: bodily fluids contain no genetic material other than that from any blood cells which may be present in lymph or blood, and thus these analyses usually require a biopsy sample from the tissue of interest. These biopsy samples may be very small, however, as the nucleic acids of interest may be amplified for detection. Thus, in many cases, the proteomic and genomic/gene expression analysis samples used will be dissimilar due to the nature of the genre of molecules to be studied.

This sort of analysis may be readily applied to complex multi-etiologic diseases, such as hepatitis. The liver is a complex multifunctional organ involved in several many synthetic, metabolic and excretory processes essential for life. It plays a major role in the regulation of carbohydrate, protein, and lipid metabolism. The liver produces most of the coagulation factors, as well as clearing activated clotting factors from the circulation. Diseases that occur in the liver include (1) infectious, e.g., viral hepatitis, (2) toxic, e.g., alcoholic hepatitis, (3) genetic, e.g. Wilson's disease, (4) immune, e.g., auto-immune hepatitis, and (5) neoplastic, e.g., hepatocellular carcinoma. Serum levels of many cytosolic, mitochondrial and membrane-associated proteins are increased in various forms of liver disease. Total serum bilirubin, protein and albumin levels, as well as the serum enzyme activities of aspartate aminotransferase (AST), alanine aminotransferase (ALT), γ-glutamyl transferase (GGT), and alkaline phosphatase (ALP) are routinely measured to help detect, diagnose, and evaluate liver disease. Serum albumin measurements are employed to assess the severity and chronicity of liver disease. The serum albumin concentration is typically decreased in individuals with chronic disease. However, the specificity of serum albumin as a diagnostic marker for chronic disease is limited because its concentration is also decreased in cases of severe acute liver or renal disease. Elevation of serum levels of AST and ALT is common in individuals with many disorders. Individuals with hepatocellular carcinoma typically have markedly elevated levels of serum ALP with lesser elevations of AST and ALT, as well as progressive elevations of serum α-fetoprotein (AFP).

This complexity of liver function and multiplicity of possible pathologies pose a significant challenge to the unequivocal assessment of liver function. Measurement of serum levels of proteins released from (but not exclusively from) the liver is at best an indirect means of evaluating function. Much more desirable would be a method for directly comparing the status of liver cells (e.g. healthy versus diseased), by determining the differential expression levels of genes specifically associated with defined metabolic or developmental states. Combining such gene expression results with proteomic profiling (measurements of specific protein gene products) would provide a much more, direct, complete and definitive assessment of liver function.

The present invention provides the means for obtaining such results on a single multiplex electronic chip wherein gene expression and immunoassays are assembled at different sites via the electronic addressing of the appropriate pRNA conjugates, and the use of electronic hybridization methodologies. For the gene expression assays, pRNA is conjugated to a DNA probe to form a pRNA-DNA chimera. This chimera is then electronically addressed to the complementary pRNA sequence previously attached to the desired locations on the chip. The unique pairing properties of pRNA assure that there is no possibility of hybridization between the pRNA and any natural DNA sequences, although carefully chosen nucleic acid sequences could also be utilizes as pairing components. Amplification and detection of mRNA levels on the active electronic matrix array has been described in detail in U.S. Ser. No. 09/710,200, filed Nov. 9, 2000, entitled “Improved Methods for Gene Expression Monitoring on Electronic Microarrays,” incorporated fully herein by reference. Shortened (50-200 bases) cDNA or RNA amplicons of the target sequence may be generated by linear amplification techniques as described in that application. These amplicons of the gene(s) of interest are then bound electronically to the p-RNA/DNA probe. Finally the amplicons detected and quantified by binding of a complementary labeled DNA reporter, or by primer extension. The group of monitored genes will typically contain so called house keeping genes, e.g, actin, or GAPDH, or an exogenous standard nucleic acid sequence for the comparison of multiple gene expression samples by correcting for any differences in cDNA amplification efficiency.

At other locations on the chip, p-RNA sequences are used as pairing components to assemble immunoassays or other protein binding assays via the addressing of antibodies or other binding elements, as described in detail above. As the individual test sites of the active electronic matrix may be individually controlled, when the immunoreaction complexes are electronically addressed separately from the these p-RNA sequences may be the same as, or different from, those used to electronically address the hybrid p-RNA/DNA capture probes utilized for the gene expression analysis, above. The use of multiple pRNA pairing components allows the multiplex determination of the expression or secretion of several gene products.

The gene expression analysis sample mixture is typically added before or after the proteomic analysis mixture, in order to avoid interference from the assays. However, as the same electronic addressing buffer and current conditions may be used, it is theoretically possible to concurrently address both the nucleic acid and immunoreaction complex portions of the assay at the same time. After electronic addressing and electronic hybridization of the assay components for each sample, and the application of hybridization probes or primer extension, the results of the assay may be detected simultaneously by fluorometry or other suitable detection means. By allowing the multiplexing of gene expression analysis and direct measurement of expressed proteins on a single chip, the present invention provides a more complete assessment of the metabolic and developmental state of the liver cell. This in turn can provide a more accurate and complete assessment of liver function. This general method may be similarly applied to other complex diagnostic problems.

### Detection and Reporting of Immuno-complex Formation in the Methods of the Invention

In order to detect and identify the formation of supra-molecular immunoreaction complexes which have formed at specific test site(s) on the array surface, it is preferred that one or more immunoreaction components be labeled with distinguishably detectable labeling moieties, or detectable moieties. Preferred reporter group(s) for use in the inventions are fluorophores. However, also suitable are chromophores, biotin/avidin detection systems, chemiluminescent agents (such as acridinium), enzymes, gold particles, magnetic beads, metal chelates, radioisotopes, other antibodies, and nanoparticles. Suitable fluorophores include active-ester or other reactive derivatives of BODIPY_{630/650} X-SE, Texas Red X-SE, or BODIPY TRX-SE, Cy-dyes, fluorescein, rhodamine, phycoerythrin, Lissamine, and coumarin. The popular Cyanine dyes such as Cy3 and Cy5 are particularly preferred for use in the methods and compositions of the invention.

By way of example, in a first immunoreaction complex, the P₁′-P₁-I₁C_{B} may be a p-RNA-antibody which is labeled with a Cyanine-3 fluorophore (Ex 530 m, Em 570 nm), the I₁C_{A} may be an antigen, and the I₁C_{L} of the immunoreaction complex sandwich may be another antibody which is labeled with a Texas Red fluorophore (Ex 590 nm, EM 620 nm). In this case, two color fluorescent analysis can be used to detect both the specific addressing of the capture antibody from the immunoreaction mixture (the Cy3 dye), and the formation of an immunoreaction complex (the Texas Red dye) on the array surface. This provides an internal experimental control to ensure that the p-RNA-antibody is properly addressed to its complementary pairing component on the test site.

Several methods of detecting such fluorescently labeled immunoreaction components in immobilized array formats are well known in the art, as are methods for detecting other types of reporter groups. Excitation/detection equipment that is able to rapidly gather fluorescence data from microarrays with 100, 400, 1000, and over 10,000 test sites in very dense packing arrangements has been described in U.S. Ser. No. 08/846,876, entitled “Scanning Optical Detection System,” filed May 1, 1997, and is suitable for use in the methods of the invention.

### EXAMPLES

The following examples are offered to further illustrate the various aspects of the present invention, and are not meant to limit the invention in any fashion. Based on these examples, and the preceding discussion of the embodiments and uses of the invention, several variations of the invention will become apparent to one of ordinary skill in the art. Such self-evident alterations are also considered to be within the scope of the present invention.

### Example 1—Synthesis and Purification of p-RNA

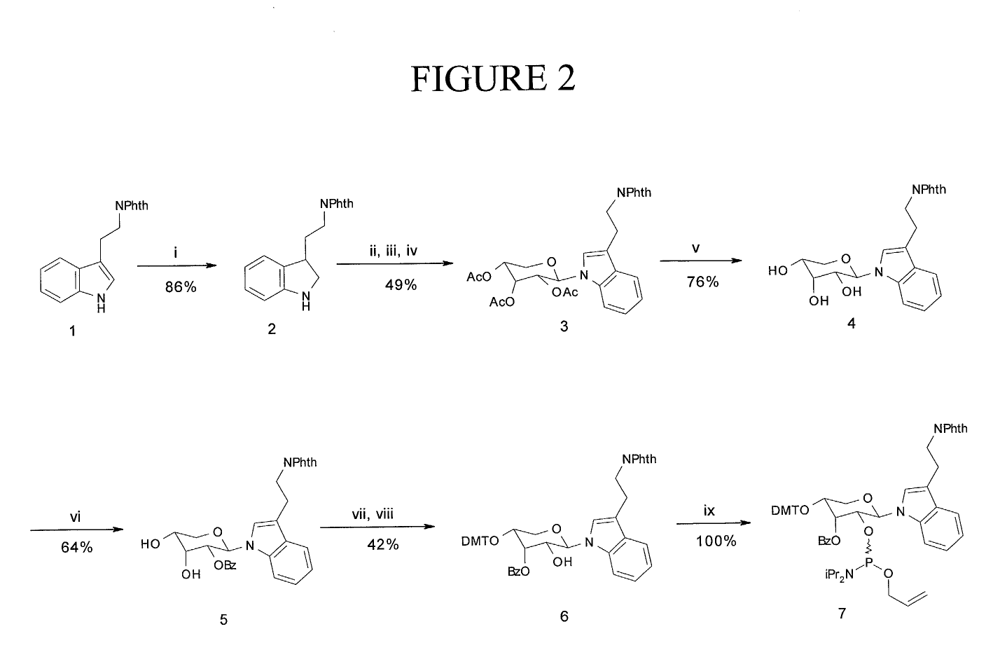

The automated synthesis of the p-RNA oligonucleotides in a typical 15 μM scale was carried out using an ECOSYN™D300⁺-Eppendorf, BIOTRONIC DNA Synthesizer. Solid support derivatives derived from DMT-protected pyranosyl-nucleoside (A,T,G,C)-precursors free at the 2′-position and benzoylated at the 3′-position where used for the p-RNA synthesis. Pyranosyl nucleotides may be synthesized by any suitable means. One useful reaction scheme for the synthesis of the β-D-ribopyranosylphosphoramidite of tryptamine from phthalyl-N-tryptamine appears in FIG. 2, wherein the reaction conditions at each step are as follows: i) 1M-borane-THF, CF₃CO₂H, 0° C., 30 min; ii) D-ribose, dry ethanol, reflux, 4h; iii) Ac₂O, py., rt, 18h; iv) DDQ, CH₂Cl₂, rt, 1.5h; v) MeONa, dry methanol, rt, 18h; vi) C₆H₅COCl, CH₂Cl₂, DMAP, py., −78° C., 15 min; vii) DMTCl, CH₂Cl₂, DMAP, py. DIPEA, molecular sieves, rt, 4.25h; viii) DMAP, py., DIPEA, n-propanol, p-nitrophenol, 75-80° C., 96h; ix) ClP(Oallyl)(iPr₂N), DIPEA, CH₂Cl₂, rt, 2h.

CPG solid support materials were used in carrying out the standard phosphoramidite p-RNA synthesis and for incorporating the tryptamine ribopyranosyl (I) phosphoramidite monomer. Phosphoramidite pyranosyl RNA nucleotide monomers, tryptamine ribopyranosyl (I) phosphoramidite monomers, as well as commercial phosphoramidite dyes (cyanine 3, cyanine 5, etc.), amino linker moieties, and biotin moieties can all be linked via a the standard succinate linker to the CPG-support. The phosphoramidite synthesis methodology used is preferably the allyl-oxy-phosphoramidite strategy described for DNA in Y. Hayakawa, S. Wakabayashi, H. Kato, R. Noyori, J. Am. Chem. Soc. 1990,112,1691.

The synthesis protocol included the following steps: (1) DMT deblocking was carried out using 6% dichloroacetic acid (v/v) in dichloromethane (100 ml); (2) washing with dichloromethane (20 ml), washing with acetonitrile (20 ml), and flushing with argon; (3) coupling by first washing the CPG solid support material with the activator (0.5 M pyridinium hydrochloride in dichloromethane (0.2 ml), then 30 minutes treatment with 1/1- activator (0.76 ml of the phosphoramidites (8 eq; 0.1 M dissolved in acetonitrile); (4) washing with acetonitrile (20 ml); (5) capping with a 2 minute treatment with 50% Cap A (10.5 ml) and 50% Cap B (10.5 ml) reagents from PerSeptive (Cap A: THF, lutidine, acetic-anhydride; Cap B: 1-methylimidazole, THF, pyridine); (6) washing with acetonitrile (20 ml); (7) the oxidizing by a 1 minute treatment with 120 ml of the oxidation solution (a freshly prepared solution of 400 mg of iodine in 100 ml of acetonitrile, adding 9.2 ml of 2,4,6-collidine and 46 ml of water.).

Before cleavage from the solid support, the p-RNA-oligonucleotide was first allyl-deprotected at the phosphotriester linkages and at the guanine bases under the conditions described by Noyori and coworkers. (Y. Hayakawa, S. Wakabayashi, H. Kato, R. Noyori, J. Am. Chem. Soc. 1990, 112, 1691). This was carried out by suspending the support in a mixture of 272 mg of Pd(PPh₃)₄, 272 mg of PPh₃ and 272 mg of Et₂NH₂HCO₃ in 15 ml of dichloromethane at room temperature. The suspension was agitated vigorously for 4 to 5 hours. The support was then carefully washed with dichloromethane (30 ml), acetone (30 ml) and water (30 ml), suspended for 30 minutes in a 0.1M solution of sodium diethyldithiocarbamate in water, and washed again with water (15 ml), acetone (15 ml) and dichloromethane (15 ml). The cleavage from the solid support and the deacylation of the bases and sugars was effected by hydrazinolysis at 4° C. within 25-40 hours (25% hydrazine hydrate in water, 6ml). Hydrazine is removed from the crude oligonucleotide by desalting over a Sep-Pak-cartridge (elution with acetonitrile/triethyl-ammonium-hydrogencarbonate 0.1M). The oligonucleotide containing fractions were combined and evaporated to dryness. After a first purification of the oligonucleotide by HPLC (LiChrospher 100 RP-18 (10μM) Merck; buffer A: triethyl-ammonium-acetate 0.1M; buffer B : triethyl-ammonium-acetate 0.1M/acetonitrile ¼) the dimethoxytrityl group at the 4′-end was cleaved off by dissolving the oligonucleotide (dry) in formic acid/water=3/2 at room temperature. After evaporation of the acid, the now fully deprotected oligonucleotide was dissolved in water and purified on a preparative HPLC-column (RP 18). The combined product fractions were evaporated to dryness and then dissolved in a 0.1 M triethyl-ammonium-hydrogencarbonate and desalted over a Sep-Pak-C18 (Waters) cartridge. The eluted product was evaporated in vacuum, once dissolved with 2 ml of water and re-evaporated to dryness and then dissolved in 1 ml of water for the determination of the optical density. For quality control, the oligonucleotide was injected on an analytical RP18 column. (>95%). The product was characterized and identified by ESI-MS.

### Example 2—Procedure for Attachment of p-RNA to Labeled Antibody Fab Fragments via Iodoacetyl Linker Chemistry

In several of the following examples, the p-RNA oligomers used as pairing component members were linked to antibody fragments utilizing a conventional iodoacetyl linkage reaction. An iodoacetylation of p-RNA with N-(iodoacetyloxy)-succinimide was carried out using 1 equivalent of the tryptamine amino-linked p-RNA-oligomer in a 0.1 molar solution of sodium-bicarbonate (pH 8.4; 1 ml per 500 nmol oligo) mixed with a solution of 70 equivalent of N-(iodoacetyloxy)-succinimide (M=283.018) in DMSO (10%=0.1 ml per 500 nmol oligo). The tube was kept in the dark at ambient temperature (25° C.) for approximately 30-90 minutes. Completion of reaction was monitored by HPLC [Buffer A: 0.1 M triethylammonium acetate in water buffer B: 0.1 M triethylammonium acetate in water: CH₃CN=1:4 method 1: starting with 10% buffer B; proceeding to 50% buffer B in 40 minutes; observation wavelength 260 nm method 2: starting with 10% buffer B; proceeding to 45% buffer B in 100 minutes; observation wavelength 260 nm on a Merck 10 μM LiChrosphere™ 100 RP-18 column; 250×4 mm. The product was eluted any where from 4 to 9 minutes after the unmodified iodoacetylated oligonucleotide. The product was desalted and further purified by standard work-up procedure on a Sep Pak™ cartridge. The solution was poured over an activated Sep Pak™ cartridge, washed with 20 ml 0.1 M TEAB buffer solution and eluted with pure acetonitrile. The product yield was determined by UV absorption at 260 nm, and then the product was lyophilized to dryness using a vacuum centrifuge.

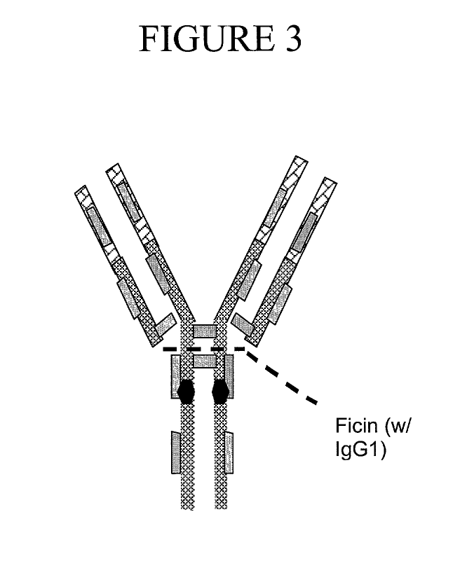

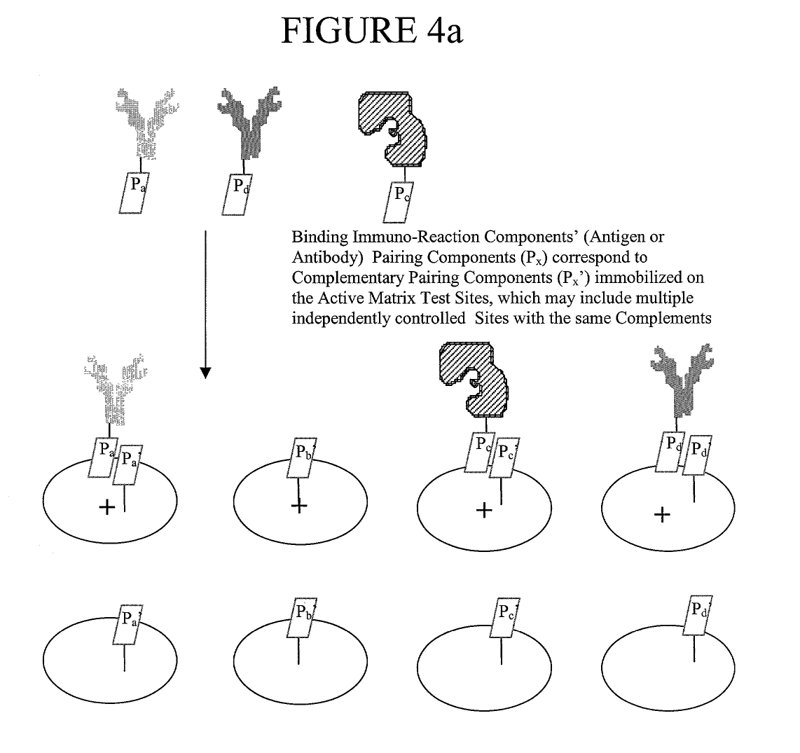

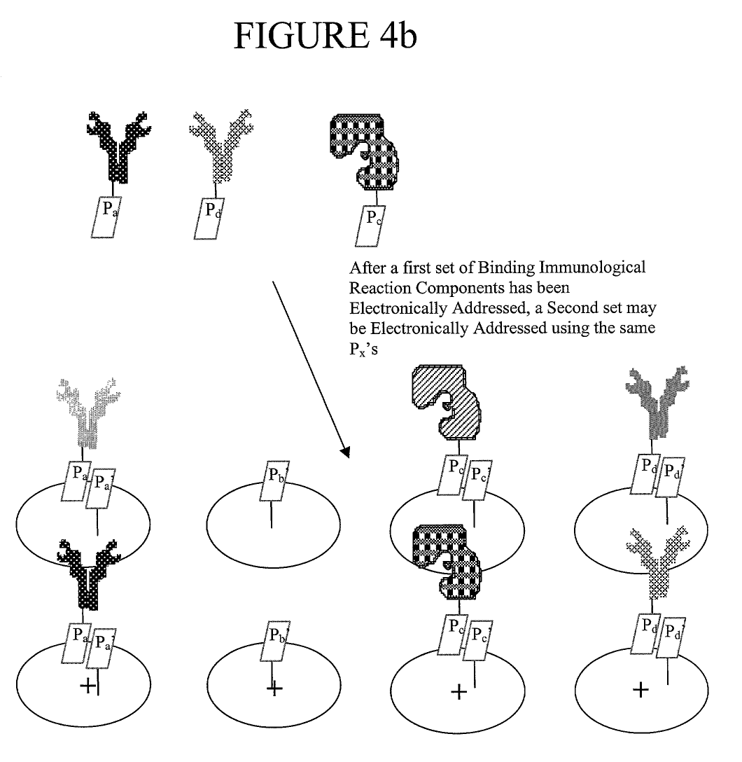

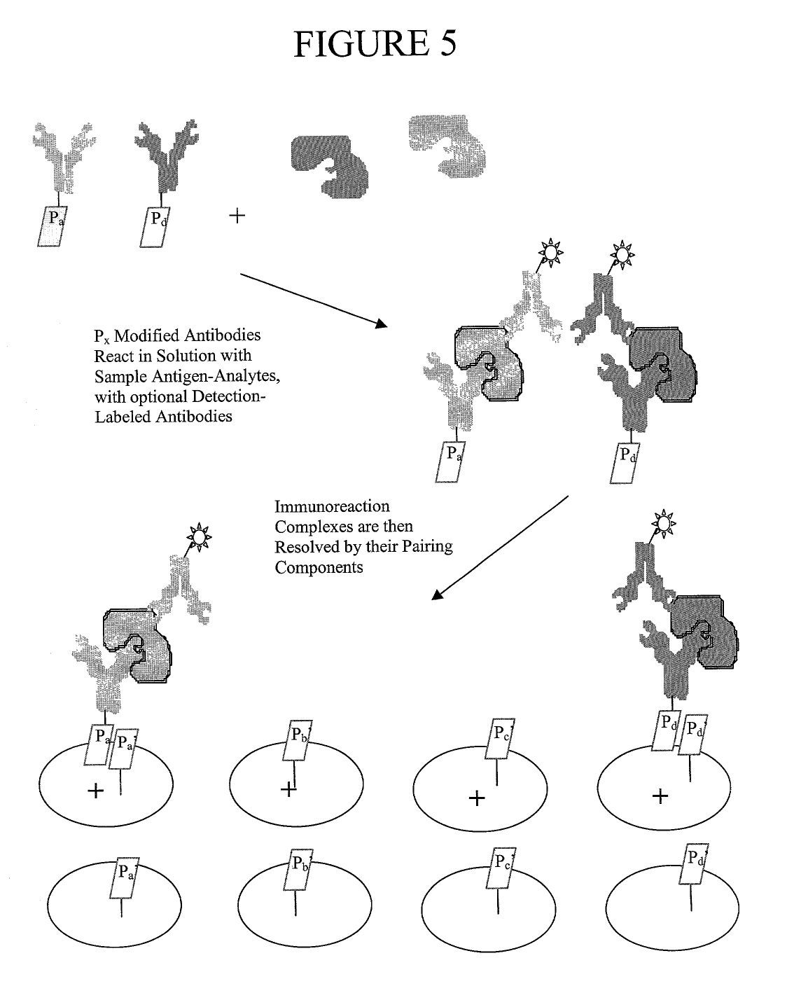

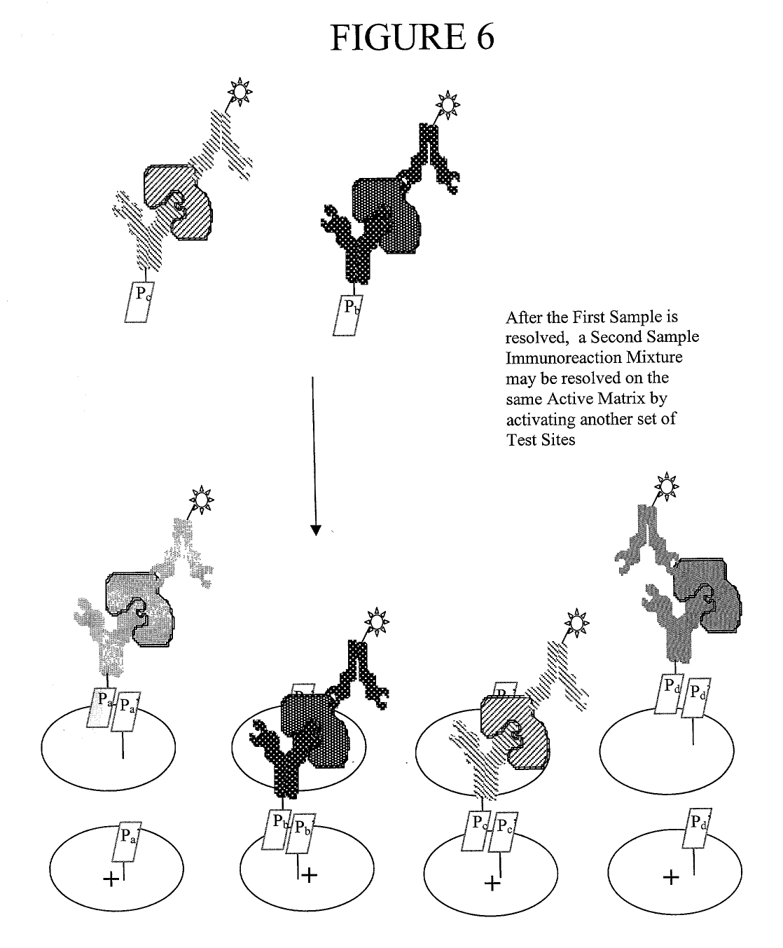

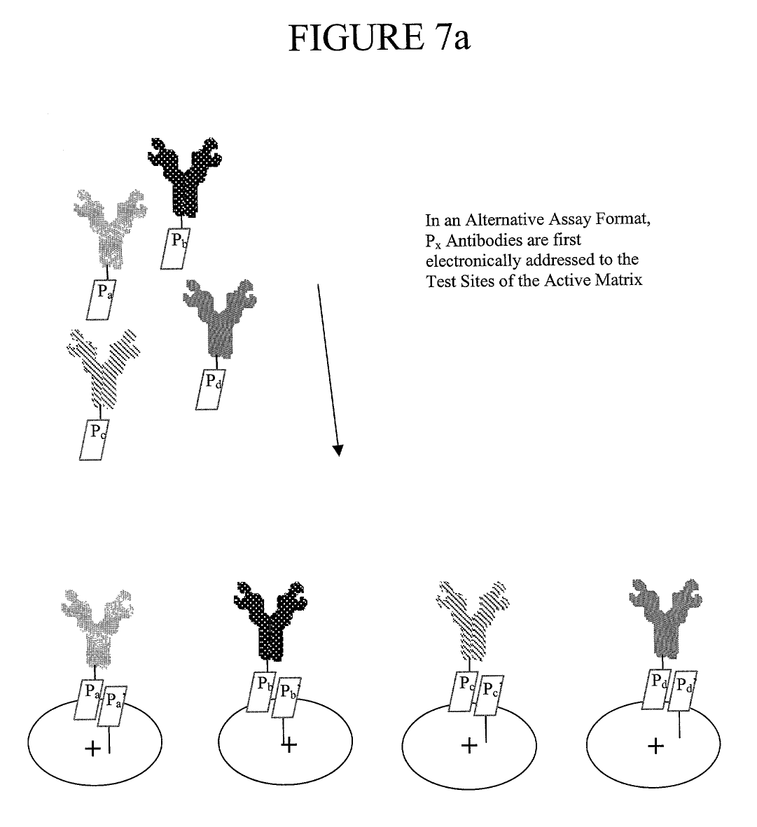

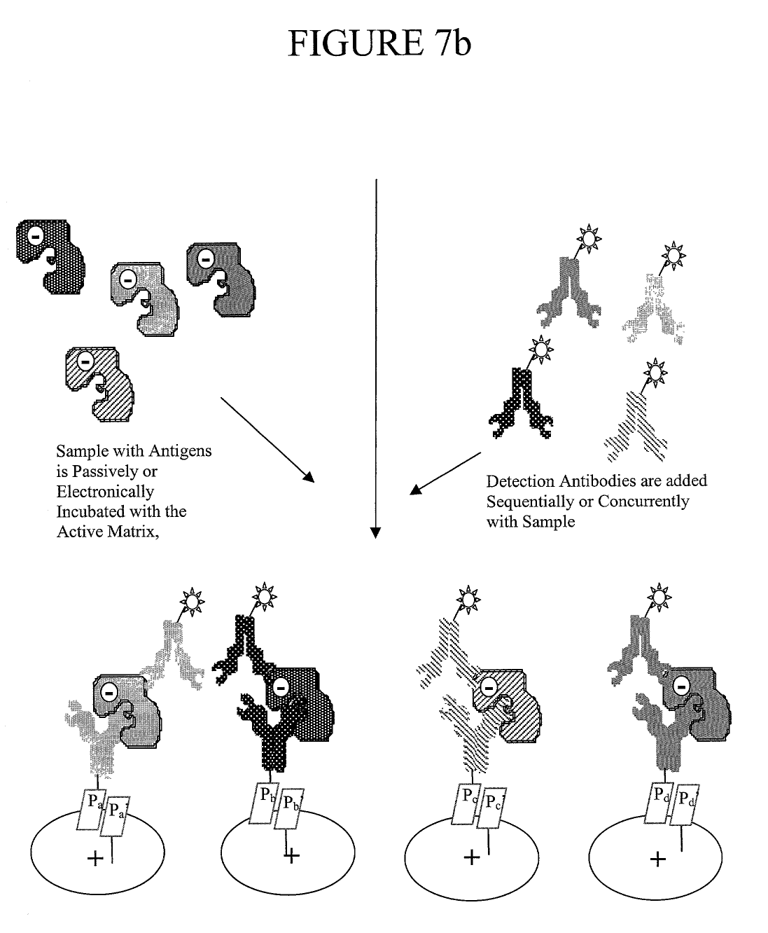

Antibodies utilized in the following examples are often digested with ficin to produce an F(ab′)₂ fragment, which consists of two epitope-binding arms (each with a portion of the light and heavy chairs of the antibody) connected by a disulfide linkage, see FIG. 3. In general, labeling and conjugation of the ficin fragment of an IgG antibody is summarized in the following scheme:

immobilized ficin, ImM cysteine Cy3-mono-succ ester IgGl * F(ab′)₂*Cy3-F(ab′)₂50 mM 13-mercaptoetbylarnine PCy3-FAb′-SH; Cy3-FAb′-SH +iodoacetyl-p-RNA -Cy3-Fab′-s-p-RNA

Alternatively, whole antibodies may be coupled to p-RNA without prior digestion. After dialyzing the antibody (1 mg/mL) against buffer composed of 20 mM phosphate, 150 mM NaCl, 1 mM EDTA, pH 7.5, the following series of additions are made: a) a volume of SPDP (N-succinimidyl 3 (2pyridyldithio) propionate, Pierce Chemical) stock (20 mM in dry acetonitrile) is slowly added under vortexing to achieve an SPDP/antibody molar ratio of 7:1, and allowed to react for 30 min at RT; b) a volume of cy3 monofunctional labeling reagent (Amersham) stock (250 μM in water) is slowly added under vortexing to achieve a cy3 reagent/antibody molar ration of 4:1 (note: following addition of the cy3 labeling reagents all steps are done under subdued lighting or in the dark to the extent possible), then a stock of 1 M sodium bicarbonate is quickly added to a final concentration of 100 mM, and the reaction is set aside for 30 min at RT; c) dithiothreitol stock (100 mM in water) is quickly added to a final concentration of 1 mM and allowed to react for 30 min at RT and in the dark. Excess reagents are removed and the buffer is exchanged by desalting the reaction mix on a G25 Sephadex column equilibrated in buffer composed of 50 mM borate, 5 mM EDTA, pH 8.3. The antibody concentration and the cy3 content of the antibody are determined by uv-visible spectroscopy; the presence of reactive sulfhydryl groups on the antibody are confirmed by assay with DTNB (Ellman's reagent, Pierce Chemical).

The cy3-labeled, sulfhydryl derivatized antibody is then divided into two aliquots: to one a volume of iodoacetyl-derivatized pRNA (IA-pRNA) stock is added to achieve a molar excess of IA-pRNA; to the other a volume of iodoacetamide stock is added to achieve the same molar excess. The two reactions—the antibody/pRNA coupling reaction and the control reaction—are left to incubate in the dark at RT overnight, after which a fresh iodoacetamide stock is added a 10-fold molar excess over antibody. Excess reagents are removed from the products by dialysis (SpectraPor microdialyzer fitted with a 25,000 or 50,000 MWCO membrane) against buffer PBS containing 20% methanol, by size-exclusion HPLC using a Zorbax GF-250 column (Agilent) and PBS buffer containing 20% methanol. Purification of the antibody-pRNA conjugate can also be purified by affinity chromatography on a resin containing all or a portion of the complementary strand of pRNA.

This protocol can be readily modified to accommodate a variety of other cross-linking reagents and approaches. A maleimide-containing pRNA could be substituted for the iodoacetyl-derivatized-pRNA if desired. On the antibody a carbodiimide could be used to introduce sulfhydryl groups via carbonyl-containing side-chains or the C-terminus instead of the amine-reactive SPDP, or the intra- and interchain disulfides of the antibody could be reduced to sulfhydryls and thereby participate as the nucleophile in the coupling reaction. Another way to introduce a nucleophile to the antibody is through carbohydrate groups using a reagent such as PDPH (3-[2-pyridyldithio]proprionyl hydrazide) from Pierce Chemical. The reverse approach to the coupling reaction would be to introduce the electrophile to the antibody by, for example, using an amine-reactive cross-linker such as SMCC (succinimidyl 4-(N-maleimidomethyl)cyclohexane-1-carboxylate) from Pierce Chemical and a sulfhydryl-derivatized pRNA.

Example 3—Demonstration of Novel Immunoreagent Immobilization Techniques via p-RNA Tethers

For this experiment the following pairs of complementary p-RNA constructs were used as pairing components for a protein conjugate consisting of Streptavidin and a goat anti-human F(ab′)₂ antibody:

#80 4′ Biotin-I-G-G-G-A-A-G-G-G-2′

#81 4′ Biotin-C-C-C-T-T-C-C-C-2′

#54 4′ Biotin-T-A-G-G-C-A-1-T-2′

#79 4′ Biotin-A-I-T-G-C-C-T-A-2′

p-RNA #81 was used to provide a capture sequence for p-RNA #80 by binding the biotin of p-RNA #81 to Streptavidin which had been immobilized in the permeation layer covering an APEX chip. The biotin of p-RNA #80 was then used to bind to a mobile streptavidin which had been chemically conjugated to a goat anti-human IgG F(ab′)₂ antibody. The goat anti-human IgG F(ab′)₂ antibody was used to capture its specific antigen target, fluorescein labeled human IgG.

Similarly a different complementary pair, p-RNAs #54 and #79, was used to form another immobilization tether. p-RNA #54 was immobilized to a permeation layer overlaying an APEX chip by binding its 4′ biotin to Streptavidin which was immobilized in the permeation layer. p-RNA #79 was hybridized to its complementary strand #54 and the 4′ biotin of #79 was used to immobilize the Streptavidin half of a solubilized conjugate of Streptavidin and goat anti-human IgG F(ab′)₂ antibody. The goat anti-human IgG F(ab′)2 antibody was then used as an immunosorbent to capture its target antigen, which is human IgG.

Selectivity of the respective capture p-RNA capture strands (either #81 or #54) was evidenced that when the complementary strands of p-RNA (either #80 or #79, respectively) were bound to the Streptavidin-goat anti-human IgG protein complex in solution and then hybridized to an APEX chip containing the captures, the p-RNAs attached to the protein conjugate bound only to their respective complementary immobilized capture strands. This demonstrates the basis for enabling geographical sorting of immunological reagents which are simultaneously present in a homogeneous solution.

### Example 4—Demonstration of Novel Methods for Achieving A Simultaneous Multiple Homogeneous Assays Combined with Discrete Analyte Detection

Using the complementary pairs of p-RNA sequences from Example 7, and including a second protein conjugate consisting of Streptavidin chemically coupled to a murine monoclonal antibody to the μ-subunit of human Chorionic Gonadotropin, simultaneous electronic addressing of two different first binding immunoreaction component-first pairing component member complexes (I₁C_{B}-P₁ and I2C_{B}-P₂) was accomplished. The p-RNA capture strands successfully differentiated between their respective complementary strands such that the two protein complexes were differentially retained by the appropriate p-RNA capture strands. Subsequent antigen detection can be accomplished by competitive means, using a labeled antigen. Optionally, a sandwich format detection may be utilized, such as where a fluorescently labeled 2^{nd} antibody binds to the antigen at a different location or epitope on the antigen than the first antibody.

This demonstrates the capability for multiple simultaneous immunological reactions to be performed in solution coupled with individual detection of the specific antigen targets of each of the individual immunological reactions. The separation of antigen target detection is accomplished by employing the selectivity of p-RNA strands for their respective complements to achieve selective antigen target detection. Detection may be by direct detection, e.g., electrical, optical or other direct detection, or by a sandwich format, such as through use of another fluorescently labeled antibody.

### Example 5—Demonstration of Novel Methods for A Multiple Analyte Assay Utilizing Electronic Incubation of Sample Analytes with Addressed Binding Immunoreaction Components

This experiment utilized two pRNA pairing components to immobilize Cy3 labeled monoclonal antibody (C_{B})-pRNA conjugates to the permeation layer on a 25-site array. Antigens were incubated separately with their respective Cy5 labeled reporter antibodies (C_{L}) and subsequently detected on the chip by electronic incubation and binding to the corresponding monoclonal antibody-pRNA conjugate. In this manner, the pRNA-antibody conjugate-antigen-reporter antibody complexes (Pₓ′-Pₓ-IₙC_{B}-IₙC_{A}-IₙC_{L}) were tethered to the array via complementary pRNA pairs.

The complementary pRNA pairs used were:

| | | |
|---|---|---|
| pRNA-1b | 4′ Biotin-TAGGCATT 2′ | (attached to perm. layer) |
| pRNA-1a | 4′ AATGCCTA 2′ | (attached to I₁C_{B}) |
| pRNA-81 | 4′ Biotin-CCCTTCCC 2′ | (attached to perm. layer) |
| pRNA-80 | 4′ GGGAAGGG 2′ | (attached to I₂C_{B}) |

The pRNA-1a (P₁) and pRNA-80 (P₂) sequences were iodoacetylated and conjugated to Cy3-labeled, SPDP derivatized monoclonal antibodies, specifically anti-CKMB (I₁C_{B}) and anti-myoglobin (anti-Mb) (I₂C_{B}) according to the protocol set forth in Example 2. The resulting conjugates were anti-Mb(Cy3)-80 (P₂-C_{B}) and anti-CKMB(Cy3)-1a (P₁-I₁C_{B}),

Reagents:

Captures (addresses 1-5)

500 nM pRNA 1b/250 nM biotin-fluorescein

500 nM pRNA 81/250 nM biotin-fluorescein

250 nM biotin-fluorescein

Cy3 labeled antibody-pRNA conjugates (addresses 7-10)

500 nM anti-Mb(Cy3)-80

500 nM anti-CKMB(Cy3)-1a

Immunoreaction Mixtures of Antigens and Cy5 labeled reporter antibodies (addresses 11-15)

250 nM myoglobin/500 nM anti-Mb′Cy5

250 nM CKMB/500 nM anti-CKMB′Cy5

250 nM myoglobin/500 nM anti-Mb′Cy5/250 nM CKMB/500 nM anti-CKMB′Cy5

Captures were diluted in 50 mM L-histidine, 0.01% Tween-20. Cy3 labeled antibody-pRNA conjugates were diluted in and dialyzed against 50 MM L-histidine, 0.01% Tween-20. Antigens and Cy5 labeled reporter antibodies were diluted in 150 mM sodium chloride, 50 mM sodium phosphate, pH=7.0 (PBS) and incubated for approximately 10 minutes at room temperature. Pre-incubated antigen and reporter antibody mixtures were then dialyzed against 50 mM L-histidine, 0.01% Tween-20. Dialysis of antibody-pRNA conjugates, antigens and reporter antibodies was performed at room temperature for 1 to 3 hours in Pierce Slide-A-Lyzer dialysis units (10,000 MWCO).

Reagents, including the immunoreaction mixtures, were electronically addressed 25-site chip according to the arrangement shown in Tables 1 and 2. Captures (addresses 1-5) were addressed at 1.8V constant voltage for 60 seconds and the array was washed 5× following each address with 15-20 μl of 50 mM L-histidine, 0.01% Tween-20 addresses 6-15 were performed at 1.8V constant voltage for 120 seconds and the array was washed 5 times with 15-20 μl of 10 mM Tris, 150 mM sodium chloride, 0.01% Tween-20, pH 7.5 following each address. Prior to electronics, pads being addressed were positively biased and ring and dump pads were negatively biased. Following all electronic addresses, the array was imaged with Cy3 and Cy5 filters.

TABLE 1
Cy3 Fluorescence Data

| | Capture addresses (1-5) | | | | | | |
|---|---|---|---|---|---|---|---|
| | 5 | 3 | | 2 | 4 | | |
| | pRNA- | pRNA- | 1 | pRNA- | pRNA- | | |
| Addresses (6-10) | 1b | 1b | histidine | 81 | 81 | Addresses (11-15) | |
| 6 50 mM Histidine | 1483 | 1554 | 778 | 1802 | 1296 | Mb/anti-Mb′Cy5/ | 11 |
| | | | | | | CKMB/anti- | |
| | | | | | | CKMB′Cy5 | |
| 7 anti-Mb(Cy3)-80 | 8101 | 6232 | 6232 | 12418 | 11550 | Mb/anti-Mb′Cy5 | 12 |
| 8 anti-Mb(Cy3)-80 | 8983 | 6219 | 6149 | 18352 | 16284 | CKMB/anti- | 13 |
| | | | | | | CKMB′Cy5 | |
| 9 anti-CKMB(Cy3)-1a | 7544 | 8001 | 1514 | 2532 | 1971 | Mb/anti-Mb′Cy5 | 14 |
| 10 anti-CKMB(Cy3)-1a | 7039 | 8265 | 1283 | 2336 | 2005 | CKMB/anti- | 15 |
| | | | | | | CKMB′Cy5 | |

TABLE 2
Cy5 Fluorescence Data

| | Capture addresses (1-5) | | | | | | |
|---|---|---|---|---|---|---|---|
| | 5 | 3 | | 2 | 4 | | |
| | pRNA- | pRNA- | 1 | pRNA- | pRNA- | | |
| Addresses (6-10) | 1b | 1b | histidine | 81 | 81 | Addresses (11-15) | |
| 6 50 mM Histidine | 1854 | 1989 | 1799 | 2425 | 2144 | Mb/anti-Mb′Cy5/ | 11 |
| | | | | | | CKMB/anti- | |
| | | | | | | CKMB′Cy5 | |
| 7 anti-Mb(Cy3)-80 | 6883 | 5800 | 5919 | 10834 | 10378 | Mb/anti-Mb′Cy5 | 12 |
| 8 anti-Mb(Cy3)-80 | 5293 | 5164 | 4016 | 4983 | 4252 | CKMB/anti- | 13 |
| | | | | | | CKMB′Cy5 | |
| 9 anti-CKMB(Cy3)-1a | 3241 | 3386 | 2756 | 3913 | 3295 | Mb/anti-Mb′Cy5 | 14 |
| 10 anti-CKMB(Cy3)-1a | 5097 | 5941 | 3126 | 3212 | 3142 | CKMB/anti- | 15 |
| | | | | | | CKMB′Cy5 | |

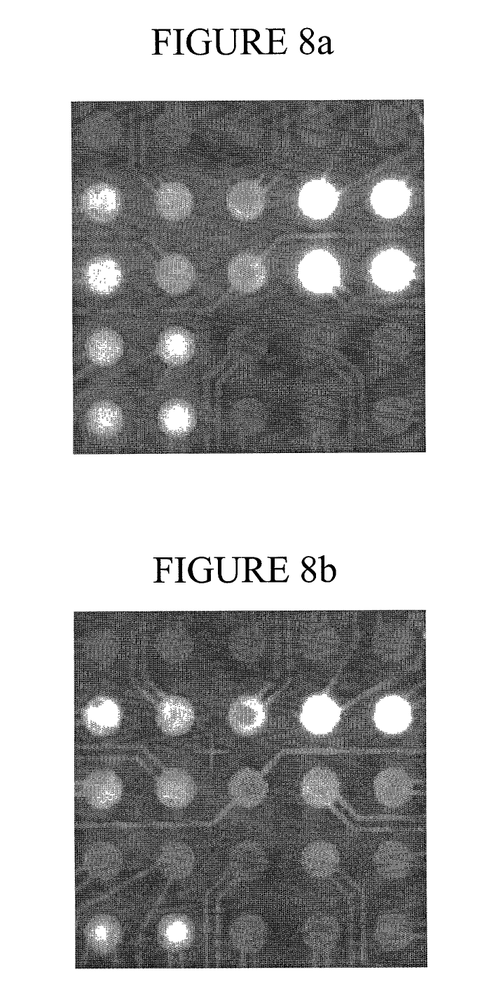

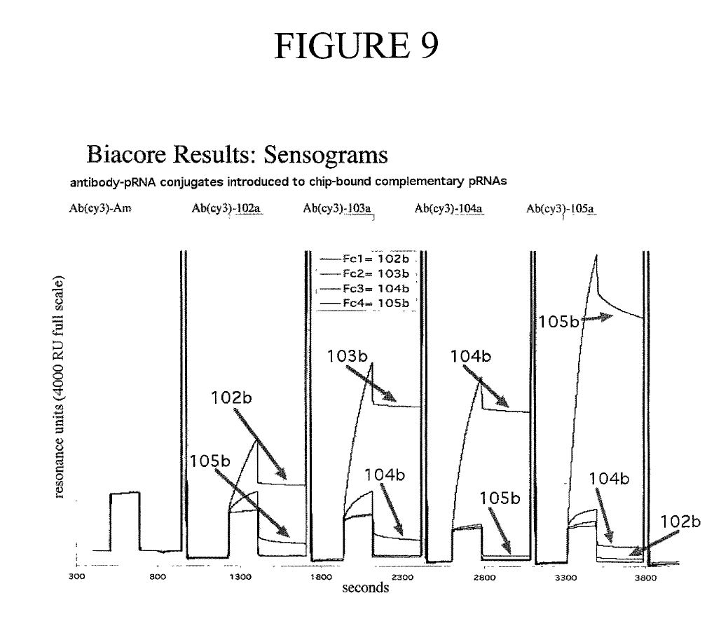

Specific binding of the antibody-pRNA conjugates and the antigen-reporter antibody complexes are readily observed in the photomicrographs, FIG. 8a (Cy3 image) and FIG. 8b (Cy5 image). After subtracting background (mean value of histidine only pads), specific to nonspecific ratios (signal on complementary pRNA capture pads to signal on non-complementary pRNA capture pads) of anti-Mb(Cy3)-80 binding approach 2 to 1 and of anti-CKMB(Cy3)-1a binding are 3.5 to 1. Specific to nonspecific ratios of anti-Mb′Cy5 and anti-CKMB′Cy5 binding are both approximately 2 to 1. This experiment demonstrates the feasibility of independent detection of distinct antigens on the same array via the electronic addressing of individual immunochemical reaction products from different sample volumes.

### EXAMPLE 6

### Demonstration of Specific Hybridization recognition by Conjugates of anti-CKMB antibody using lodoacetylated-C 6-spacer pRNA and SPDP-derivatized, cy3-labeled intact Mab

p-RNA/antibody conjugates were prepared using intact IgG1 antibodies and pRNA 10-mers. The “a” series of p-RNA oligomers were conjugated to the antibody through an n-hexyl spacer at the 4′ end of the p-RNA. Each intact monoclonal CKMB antibody was first modified with SPDP to generate free sulfhydryl groups (10 -SH per antibody) and labeled with cy3 (2.6 cy3 per antibody). The conjugates were prepared by reacting the modified antibodies with the iodoacetyl-C6-100a series oligomers. A negative control was prepared in a reaction in which iodoacetamide was substituted for pRNA. After an overnight incubation at room temperature the remaining sulfhydryl sites on the conjugates were capped with iodoacetamide and purified by size exclusion-HPLC. The fractions containing the conjugates (anti-CKMB(cy3)-pRNA or anti-CKMB(cy3)-acetamide control) were concentrated using 30,000 M.W. cut-off filtration devices.

Calculations based on the chromatograms recorded at 260 nm and 550 nm during the purification steps yielded the following degrees of pRNA incorporation in the conjugates based on the anti-CKMD(cy3)-acetamide control: anti-CKMB(cy3)-102a, 1.0:1; anti-CKMB(cy3)-103a, 2.0:1; anti-CKMB(cy3)-104a, 1.7:1; anti-CKMB(cy3)-105a, 1.3:1.

Hybridization of the anti-CKMB(cy3)-p-RNA conjugates to their corresponding capture pRNA sequences was assessed in Biacore-instrument experiments in which each of the conjugates was injected through four flow cells. Each flow cell of the instrument contained a gold surface covered with a streptavidin-agarose gel layer, to which one of the capture p-RNAs (i.e. 102b, 103b, 104b, and 105b) bound at the Sensor Chip surface by a streptavidin-biotin interaction. In this way match and mismatch binding of a conjugate was simultaneously monitored.

The results of the Biacore experiments are shown in the following table, which shows mismatch binding by each capture relative to its match conjugate. The results are also shown in FIG. 10.

TABLE 6
Capture cross-reactivity, %

| | 102b | 103b | 104b | 105b |
|---|---|---|---|---|
| Anti-CKMB (cy3)-102a | 100.0 | 1.5 | 1.6 | 7.1 |
| Anti-CKMB (cy3)-103a | 3.6 | 100.0 | 16.4 | 1.5 |
| Anti-CKMB (cy3)-104a | 1.6 | 1.7 | 100.0 | 2.1 |
| Anti-CKMB cy3)-105a | 6.4 | 1.1 | 11.2 | 100.0 |

These results show that antibodies conjugated to p-RNA can be addressed specifically to sites on a chip surface that contain the complementary p-RNA sequence. Although some “cross-talk” is evident in the data, for example the antibody-p-RNA 103a conjugate shows 16.4% binding to the non-matching sequence 104b, this is a reflection of non-specific binding of the antibody. Biacore Sensorgrams of the same pRNA sequences devoid of protein do not exhibit any significant cross-talk.

Although the foregoing invention has been described in some detail by way of illustration and example for purposes of clarity and understanding, it may be readily apparent to those of ordinary skill in the art in light of the teachings of this invention that certain changes and modifications may be made thereto without departing from the spirit or scope of the appended claims.

What is claimed is:

1. A method of detecting the presence of one or more analytes (I₁C_{A}, . . . , IₙC_{A}) in a sample, the method comprising:
a) contacting a sample with a set of binding immunoreaction component-pairing component member complexes (I₁C_{B}-P₁, . . . , IₙC_{B}-Pₓ), wherein the set comprises at least one binding immunoreaction component-pairing component member complex for each analyte to be detected, producing immunoreaction complexes (I₁C_{A}-I₁C_{B}-P₁, . . . , IₙC_{A}-IₙC_{B}-Pₓ) of the analytes in the sample and their respective binding immunoreaction component-first pairing component member complexes;
b) contacting the complexes formed in step (a) with an active electronic matrix, wherein the active electronic matrix comprises at least one set of test sites, wherein the set comprises at least one test site comprising a complementary pairing component member attached the test site's permeation layer for each pairing component member of in the set of binding immunoreaction component-pairing component member complexes of step (a);
c) electronically biasing, concurrently or sequentially, the set of test sites to which the complementary pairing components are attached, whereby the pairing component members on the immunoreaction complexes and on the test sites are allowed to bind, producing a bound immunoreaction complexes at the biased test sites;
d) detecting the presence of the bound immunoreaction complexes at the test sites.

2. The method of claim 1 wherein at least one analyte is an epitope bearing moiety.

3. The method of claim 1 wherein at least one analyte is a moiety selected from the group consisting of: immunoglobulins, recombinant immunoglobulins, single-stranded engineered immunoglobulins humanized immunoglobulins, hybridized immunoglobulins, immunoglobulin derivatives, and immunoglobulin fragments.

4. The method of claim 1 wherein at least one binding immunoreaction component in the set of binding immunoreaction component-pairing component member complexes is an epitope bearing moiety.

5. The method of claim 1 wherein at least one binding immunoreaction component in the set of binding immunoreaction component-pairing component member complexes is a moiety selected from the group consisting of: immunoglobulins, recombinant immunoglobulins, single-stranded engineered immunoglobulins humanized immunoglobulins, hybridized immunoglobulins, immunoglobulin derivatives, and immunoglobulin fragments.

6. The method of claim 1 wherein at least one of the pairing components is p-RNA.

7. The method of claim 1 wherein at least one of the pairing components is CNA.

8. The method of claim 1 wherein at least one of the pairing components is DNA.

9. The method of claim 1 wherein at least one of the pairing components is RNA.

10. The method of claim 1 wherein at least one of the binding immunoreaction components is attached to its respective pairing component member through a covalent bond.

11. The method of claim 1 wherein at least one of the binding immunoreaction components is attached to its respective pairing component member through a non-covalent interaction.

12. The method of claim 11 wherein the non-covalent interaction is selected from the group consisting of a biotin-streptavidin interaction and a biotin-avidin interaction.

13. The method of claim 1 wherein at least one complementary pairing component member is attached to its test site through a covalent bond to the permeation layer.

14. The method of claim 1 wherein at least one complementary pairing component member is attached to the test site through a non-covalent interaction with a moiety in the permeation layer.

15. The method of claim 14 wherein the non-covalent interaction is selected from the group consisting of a biotin-streptavidin interaction and a biotin-avidin interaction.

16. The method of claim 1 wherein one analyte (I₁C_{A}) is detected.

17. The method of claim 1 wherein at least two analytes (I₁C_{A}, I₂C_{A}) are detected.

18. The method of claim 1 wherein at least three analytes (I₁C_{A}, I₂C_{A}, I₃C_{A}) are detected.

19. The method of claim 1 wherein at least four analytes (I₁C_{A}, I₂C_{A}, I₃C_{A}, I₄C_{A}) are detected.

20. The method of claim 1 wherein at least five analytes (I₁C_{A, I}₂C_{A}, I₃C_{A}, I₄C_{A, I}₅C_{A}) are detected.

21. The method of claim 1 wherein the active electronic matrix array comprises at least a second set of test sites, wherein each set comprises at least one test site comprising a complementary pairing component member attached the test site's permeation layer for each pairing component member of in the set of binding immunoreaction component-pairing component member complexes of step (a), wherein the second set of test sites is not electronically biased in step (c).

22. The method of claim 21 further comprising repeating steps (a) through (c) with at least one additional sample, wherein in step (c) of each repeat cycle, for each sample, a set of test sites which was not biased during step (c) for any of the other samples is biased so that the pairing component members on the immunoreaction complexes and on the biased test site are allowed to bind, producing bound immunoreaction complexes on the biased test sites for that sample.

23. The method of claim 22 wherein two additional samples are processed so as to produce bound immunoreaction complexes for each sample.

24. The method of claim 22 wherein three additional samples are processed so as to produce bound immunoreaction complexes for each sample.

25. The method of claim 22 wherein four additional samples are processed so as to produce bound immunoreaction complexes for each sample.

26. The method of claim 22 wherein at least nine additional samples are processed so as to produce bound immunoreaction complexes for each sample.

27. The method of claim 1 further comprising contacting the sample with at least one labeled analyte standard, wherein the labeled analyte standard comprises a detectable moiety and a moiety selected from the group consisting of the analyte and an immunochemical analog of the analyte, wherein the detection of the presence of the bound immunoreaction complex containing the analyte in step (d) is by detecting the decrease in the amount of the immunoreaction complex containing the labeled analyte standard bound at the test site.

28. The method of claim 27 wherein the labeled analyte standard comprises the analyte.

29. The method of claim 27 wherein the analyte is an antibody, and the labeled analyte standard comprises an analog of the analyte comprising an isoidiotypic binding site, wherein the analyte analog is selected from the group consisting of: immunoglobulins, recombinant immunoglobulins, single-stranded engineered immunoglobulins humanized immunoglobulins, hybridized immunoglobulins, immunoglobulin derivatives, and immunoglobulin fragments.

30. The method of claim 27 wherein the analyte is an antigen, and the labeled analyte standard comprises an analog of the analyte comprising a structural domain selected from the group consisting of: an epitope of the antigen recognized by the binding immunoreaction component which binds the analyte, and a structural moiety which is anti-idiotypic to the binding site of the binding immunoreaction component which binds the analyte.

31. The method of claim 27 wherein the labeled analyte standard comprises a detectable moiety selected from the group consisting of: fluorophores, chromophores, biotin, avidin, streptavidin, chemiluminescent agents, enzymes, gold particles, magnetic beads, metal chelates, radioisotopes, other antibodies, and nanoparticles.

32. The method of claim 27 wherein the labeled analyte standard comprises a fluorophore selected from the group consisting of: BODIPY_{630/650} X-SE, Texas Red X-SE, or BODIPY TRX-SE, Cy-dyes, fluorescein, rhodamine, phycoerythrin, Lissamine, and coumarin.

33. The method of claim 1 further comprising allowing one or more labeling immunoreaction components to bind to one or more of the analytes to be detected, wherein at least one of the labeling immunoreaction components comprises a detectable moiety, and wherein the detection of the presence of bound immunoreaction complexes at the test sites in step (d) is by the detection of the labeling immunoreaction components bound to the analytes in the bound immunoreaction complexes.

34. The method of claim 33 wherein at least one of the labeling immunoreaction components comprises a moiety selected from the group consisting of: immunoglobulins, recombinant immunoglobulins, single-stranded engineered immunoglobulins humanized immunoglobulins, hybridized immunoglobulins, immunoglobulin derivatives, and immunoglobulin fragments.

35. The method of claim 33 wherein at least one of the labeling immunoreaction components comprises a moiety selected from the group consisting of biotin, avidin, and streptavidin.

36. The method of claim 33 wherein at least one of the labeling immunoreaction components comprises a detectable moiety fluorophores, chromophores, biotin, avidin, streptavidin, chemiluminescent agents, enzymes, gold particles, magnetic beads, metal chelates, radioisotopes, other antibodies, and nanoparticles.

37. The method of claim 33 wherein at least one of the labeling immunoreaction components comprises a fluorophore selected from the group consisting of: BODIPY_{630/650} X-SE, Texas Red X-SE, or BODIPY TRX-SE, Cy-dyes, fluorescein, rhodamine, phycoerythrin, Lissamine, and coumarin.

38. The method of claim 33 wherein at least one of the labeling immunoreaction components binds to more than one analyte in the sample.

39. The method of claim 33 wherein at least one of the labeling immunoreaction components is a dye with an affinity for one or more of the analytes to be detected greater than its affinity for any other component of the immunoreaction complex or the permeation layer of the active electronic matrix.

40. The method of claim 33 wherein at least one of the labeling immunoreaction components is contacted with the sample in step (a).

41. The method of claim 33 wherein at least one of the labeling immunoreaction components is contacted with the immobilized immunoreaction complexes produced in step (c).

42. The method of claim 41 further comprising electronically biasing one or more sites containing immobilized immunoreaction complexes while contacting the at least one labeling immunoreaction component with the immobilized immunoreaction complexes.

43. A method for detecting one or more analytes (I₁C_{A}, . . . , IₙC_{A}) in a sample, the method comprising:
a) contacting with an active electronic matrix array, either concurrently or sequentially, a set of binding immunoreaction component-pairing component member complexes (I₁C_{B}-P₁, . . . , IₙC_{B}-Pₓ), the set comprising a binding immunoreaction component-pairing component member complex for each analyte to be detected, wherein the active electronic matrix array comprises at least one set of test sites, the set consisting of at least one test site comprising a complementary pairing component member for each binding immunoreaction component-pairing component member complex;
b) electronically biasing, either concurrently or sequentially, each test site to which one of the binding immunoreaction component-pairing component member complexes is to be attached, so that the pairing component members are allowed to selectively bind, thus producing a set of attached binding immunoreaction component-pairing component member complexes (I₁C_{B}-P₁-P₁′, . . . ,IₙC_{B}-Pₓ-Pₓ′) bound to the set of test sites;
c) contacting the sample with the active electronic matrix array, and incubating the sample with the array under conditions suitable for the binding of the analytes in the sample with the immobilized binding immunoreaction components, thereby producing immobilized immunoreaction complexes at one or more test sites;
d) detecting the presence of bound analyte in the immobilized immunoreaction complexes at the test sites.

44. The method of claim 43 wherein one or more analyte is an epitope bearing moiety.

45. The method of claim 43 wherein one or more analyte is a moiety selected from the group consisting of: immunoglobulins, recombinant immunoglobulins, single-stranded engineered immunoglobulins humanized immunoglobulins, hybridized immunoglobulins, immunoglobulin derivatives, and immunoglobulin fragments.

46. The method of claim 43 wherein one or more binding immunoreaction component in the set of binding immunoreaction component-pairing component member complexes is an epitope bearing moiety.

47. The method of claim 43 wherein one or more binding immunoreaction component in the set of binding immunoreaction component-pairing component member complexes is an epitope bearing moiety is a moiety selected from the group consisting of: immunoglobulins, recombinant immunoglobulins, single-stranded engineered immunoglobulins humanized immunoglobulins, hybridized immunoglobulins, immunoglobulin derivatives, and immunoglobulin fragments.

48. The method of claim 43 wherein at least one pairing component is p-RNA.

49. The method of claim 43 wherein at least one pairing component is CNA.

50. The method of claim 43 wherein at least one pairing component is DNA.

51. The method of claim 43 wherein at least one pairing component is RNA.

52. The method of claim 43 wherein at least one of the binding immunoreaction components is attached to its respective pairing component member through a covalent bond.

53. The method of claim 43 wherein at least one of the binding immunoreaction components is attached to its respective pairing component member through a non-covalent interaction.

54. The method of claim 53 wherein the non-covalent interaction is selected from the group consisting of a biotin-streptavidin interaction and a biotin-avidin interaction.

55. The method of claim 43 wherein each complementary pairing component member is attached to its respective test site through a covalent bond to the permeation layer.

56. The method of claim 43 wherein each complementary pairing component member is attached to the test site through a non-covalent interaction with a moiety in the permeation layer.

57. The method of claim 56 wherein the non-covalent interaction is selected from the group consisting of a biotin-streptavidin interaction and a biotin-avidin interaction.

58. The method of claim 43 wherein one analyte (I₁C_{A}) is detected.

59. The method of claim 43 wherein at least two analytes (I₁C_{A}, I₂C_{A}) are detected.

60. The method of claim 43 wherein at least three analytes (I₁C_{A}, I₂C_{A}, I₃C_{A}) are detected.

61. The method of claim 43 wherein at least four analytes (I₁C_{A}, I₂C_{A}, I₃C_{A}, I₄C_{A}) are detected.

62. The method of claim 43 wherein at least five analytes (I₁C_{A}, I₂C_{A}, I₃C_{A}, I₄C_{A}, I₅C_{A}) are detected.

63. The method of claim 43 wherein at least ten analytes (I₁C_{A}, . . . , I₁₀C_{A}) are detected.

64. The method of claim 43 wherein the set of binding immunoreaction component-pairing component member complexes is contacted with the active electronic matrix array concurrently, wherein all test sites to which the binding immunoreaction component-pairing component member complexes of the set are to be bound are electronically biased concurrently, and wherein each immobilized binding immunoreaction component is immobilized with a different pairing component.

65. The method of claim 43 wherein at least some of the set of binding immunoreaction component-pairing component member complexes are contacted with the active electronic matrix array sequentially, wherein at least some of test sites to which the binding immunoreaction component-pairing component member complexes of the set are to be bound are electronically biased sequentially, and wherein each immobilized binding immunoreaction component is immobilized with a different pairing component.

66. The method of claim 43 wherein at least some of the set of binding immunoreaction component-pairing component member complexes are contacted with the active electronic matrix array sequentially, wherein at least some of the test sites to which the binding immunoreaction component-pairing component member complexes of the set are to be bound are electronically biased sequentially, and wherein two or more immobilized binding immunoreaction component are immobilized with the same pairing component.

67. The method of claim 43 wherein the active electronic matrix array comprises at least two sets of test sites, each set comprising at least one test site comprising a complementary pairing component member for each binding immunoreaction component-pairing component member complex, and wherein in step (b) at least two sets of attached binding immunoreaction component-pairing component member complexes IₙC_{B}-Pₓ-Pₓ′) bound to each set of test sites are created.

68. The method of claim 43 wherein the sample is incubated with the active matrix array without electronic biasing of any of the test sites on the array.

69. The method of claim 43 wherein the sample is incubated with the active matrix array while electronic biasing one or more of the test sites on the array.

70. The method of claim 69 further comprising the incubation of at least second sample containing analytes with the active electronic matrix array after incubating the first sample with the active electronic matrix array, wherein a first set of test sites comprising a first set of attached binding immunoreaction component-pairing component member complexes is electronically biased during the incubation of the first sample with the array, and a separate set of test sites comprising another set of attached binding immunoreaction component-pairing component member complexes is electronically biased during the incubation of the each subsequent sample with the array.

71. The method of claim 43 further comprising contacting the active matrix array with a labeled analyte standard for at least one of the analytes to be detected, wherein each labeled analyte standard comprises a detectable moiety and a moiety selected from the group consisting of the corresponding analyte and an immunochemical analog of the corresponding analyte, wherein the detection of the presence of bound analyte in the immobilized immunoreaction complexes at the test sites in step (d) is by detecting the decrease in the amount of bound labeled analyte standard in the immobilized immunoreaction complexes at the test sites.

72. The method of claim 71 wherein the labeled analyte standard comprises the analyte.

73. The method of claim 71 wherein the analyte is an antibody, and the labeled analyte standard comprises an analyte analog comprising an isoidiotypic binding site, wherein the analyte analog is selected from the group consisting of: immunoglobulins, recombinant immunoglobulins, single-stranded engineered immunoglobulins humanized immunoglobulins, hybridized immunoglobulins, immunoglobulin derivatives, and immunoglobulin fragments.

74. The method of claim 71 wherein the analyte is an antigen, and the labeled analyte standard comprises an analyte analog comprising a structural domain selected from the group consisting of an epitope of the antigen recognized by the binding immunoreaction component which binds the analyte and a structural moiety which is anti-idiotypic to the binding site of the binding immunoreaction component which binds the analyte.

75. The method of claim 71 wherein the labeled analyte standard comprises a detectable moiety selected from the group consisting of: fluorophores, chromophores, biotin, avidin, streptavidin, chemiluminescent agents, enzymes, gold particles, magnetic beads, metal chelates, radioisotopes, other antibodies, and nanoparticles.

76. The method of claim 71 wherein the labeled analyte standard comprises a fluorophore selected from the group consisting of: BODIPY_{630/650} X-SE, Texas Red X-SE, or BODIPY TRX-SE, Cy-dyes, fluorescein, rhodamine, phycoerythrin, Lissamine, and coumarin.

77. The method of claim 71 wherein the labeled analyte standard is contacted with the active matrix array before the sample is incubated with the active electronic matrix array in step (c).

78. The method of claim 71 wherein the labeled analyte standard is contacted with the active matrix array after the sample is incubated with the active electronic matrix array in step (c).

79. The method of claim 71 wherein the labeled analyte standard is contacted with the active matrix array while the sample is incubated with the active electronic matrix array in step (c).

80. The method of claim 71 wherein the labeled analyte standard is contacted with the active matrix without electronic biasing of any of the test sites on the array.

81. The method of claim 71 wherein the labeled analyte standard is contacted with the active matrix while electronically biasing one or more of the test sites on the array.

82. The method of claim 43 further comprising allowing one or more labeling immunoreaction components to bind to one or more of the analytes, wherein at least one of the labeling immunoreaction components comprises a detectable moiety, and wherein the detection of the presence of at least one bound analyte in immobilized immunoreaction complexes at the test site in step (d) is by detecting labeling immunoreaction components bound to the analyte.

83. The method of claim 82 wherein at least one of the labeling immunoreaction components comprises a moiety selected from the group consisting of: immunoglobulins, recombinant immunoglobulins, single-stranded engineered immunoglobulins humanized immunoglobulins, hybridized immunoglobulins, immunoglobulin derivatives, and immunoglobulin fragments.

84. The method of claim 82 wherein at least one of the labeling immunoreaction components comprises a moiety selected from the group consisting of biotin, avidin, and streptavidin.

85. The method of claim 82 wherein at least one of the labeling immunoreaction components comprises a detectable moiety selected from the group consisting of fluorophores, chromophores, biotin, avidin, streptavidin, chemiluminescent agents, enzymes, gold particles, magnetic beads, metal chelates, radioisotopes, other antibodies, and nanoparticles.

86. The method of claim 82 wherein at least one of the labeling immunoreaction components comprises a fluorophore selected from the group consisting of: BODIPY_{630/650} X-SE, Texas Red X-SE, or BODIPY TRX-SE, Cy-dyes, fluorescein, rhodamine, phycoerythrin, Lissamine, and coumarin.

87. The method of claim 82 wherein at least one of the labeling immunoreaction components binds to more than one analyte in the sample.

88. The method of claim 82 wherein at least one of the labeling immunoreaction components is a dye with an affinity for one or more analytes greater than its affinity for any other component of the immunoreaction complex or the permeation layer of the active electronic matrix.

89. The method of claim 82 wherein at least one of the labeling immunoreaction components is contacted with the sample prior to the incubation of the sample with the active electronic matrix array in step (c).

90. The method of claim 82 wherein at least one of the labeling immunoreaction components is contacted with the sample during the incubation of the sample with the active electronic matrix array in step (c).

91. The method of claim 82 wherein at least one of the labeling immunoreaction components is contacted with the immobilized immunoreaction complexes produced in step (c).

92. The method of claim 43 further comprising electronically biasing one or more sites containing immobilized immunoreaction complexes while contacting the at least one labeling immunoreaction component with the immobilized immunoreaction complexes.

93. A method for performing multiple immunological reactions on a active electronic matrix array device comprising the steps of:
a) providing a plurality of microelectronic sites, each site having a complementary pairing component member couple thereto, the complementary pairing component members varying from site to site,
b) providing a plurality of different types of antibodies, each type of antibody being labeled with a different pairing component member, the different pairing components being complementary to the pairing components coupled to the microelectronic sites,
c) providing at least one antigen in a sample,
d) contacting the sample and the plurality of labeled, different types of antibodies with the pairing components at the microelectronic sites,
e) interacting the antibody-bound pairing component member and the test-site bound complementary pairing component member, and
f) determining the sites at which an antibody coupled to the antigen coupled to the complementary pairing component is present.

94. The method of claim 93 wherein the antigen and plurality of labeled, different types of antibodies are provided together in a homogeneous format.

95. The method of claim 93 wherein the pairing component members and their corresponding complement pairing component members are p-RNA.

96. A kit for use in detecting a set one or more of analytes (I₁C_{A}, . . . , IₙC_{A}) in a sample, the kit comprising a set of binding immunoreaction component-pairing component complexes (I₁C_{B}-P₁, . . . , IₙC_{B}-Pₓ).

97. The kit of claim 96 wherein at least one of the binding immunoreaction components in the complexes of the set is an epitope bearing moiety.

98. The kit of claim 96 wherein at least one of the binding immunoreaction components in the complexes of the set is a moiety selected from the group consisting of: immunoglobulins, recombinant immunoglobulins, single-stranded engineered immunoglobulins humanized immunoglobulins, hybridized immunoglobulins, immunoglobulin derivatives, and immunoglobulin fragments.

99. The kit of claim 96 wherein the pairing component portions of the complexes are P-RNA.

100. The kit of claim 96 wherein the pairing component portions of the complexes are CNA.

101. The kit of claim 96 wherein the pairing component portions of the complexes are DNA.

102. The kit of claim 96 wherein the pairing component portions of the complexes are RNA.

103. The kit of claim 96 wherein at least one binding immunoreaction component is attached to its pairing component member through a covalent bond.

104. The kit of claim 96 wherein at least one binding immunoreaction component is attached to its pairing component member through a non-covalent interaction.

105. The kit of claim 104 wherein the non-covalent interaction is selected from the group consisting of a biotin-streptavidin interaction and a biotin-avidin interaction.

106. The kit of claim 96 further comprising a set of complementary pairing component members (P₁′, . . . , Pₓ′), wherein the complementary pairing component members are functionalized for attachment to the test sites of an active electronic matrix device.

107. The kit of claim 106, wherein the complementary pairing component members are P-RNA.

108. The kit of claim 106, wherein the complementary pairing component members are CNA.

109. The kit of claim 106, wherein the complementary pairing component members are DNA.

110. The kit of claim 106, wherein the complementary pairing component members are RNA.

111. The kit of claim 96 further comprising at least one labeled analyte standard which is bound by at least one of the binding immunoreaction component-pairing component complexes in the set.

112. The kit of claim 96 further comprising at least one labeling immunoreaction component which binds to at least one of the analytes to be detected with the kit.
